SEQUENCE LISTING (5 sequences)

SEQ ID NO: 1 - DNA, 4945 bp, Mus musculus
gcccgctgcagatcccgtaagacgggagtctgcggagtcgcttcagtccccgccgccgccacattcaacaggcagcagcgccgctgtcgcgcggccgcggagagctagagcggcccgcagcgtccgcccgtctgccttggcgtccgcggcccttgtcagcgggagcgcggtgcccgagctgccgggctccgcggcctggtcggtgccccgtcctaggcacgaactcggaggctccttagacaccggtgacccagcgaagttaagttctgggcgcgtccgtccgctgcgccccgccgcgcctgactccggcgtgcgtccgccgtccgcggccccccaatctcggagcgacactcgggtcgcccgctccgcgcccccggtggccgcgtctcccggtacttctctgctggtgggggaggggcgggggcaccatggccgaagcgccccaggtggtggagaccgacccggacttcgagccgctgccccggcagcgctcctgtacctggccgctgcccaggccggagtttaaccagtccaactcgaccacctccagtccggcgccgtcgggcggcgcggccgccaaccccgacgccgcggcgagcctggcctcggcgtccgctgtcagcaccgactttatgagcaacctgagcctgctggaggagagtgaggacttcgcgcgggcgccaggctgcgtggccgtggcggcggcggctgcggccagcaggggcctgtgcggggacttccagggccccgaggcgggctgcgtgcacccagcgccgccacagcccccaccgaccgggccgctgtcgcagcccccacccgtgcctccctccgctgccgccgccgcggggccactcgcgggacagccgcgcaagaccagctcgtcgcgccgcaacgcgtggggcaacctgtcgtacgccgacctcatcaccaaggccatcgagagctcagccgagaagaggctcaccctgtcgcagatctacgagtggatggtgaagagcgtgccctacttcaaggataagggcgacagcaacagctcggcgggctggaagaattcaattcgccacaatctgtcccttcacagcaagtttattcgagtgcagaatgaaggaactggaaagagttcttggtggatgctcaatccagagggaggcaagagcggaaaatcaccccggagaagagctgcgtccatggacaacaacagtaaatttgctaagagccgagggcgggctgctaagaaaaaagcatctctccagtctgggcaagagggtcctggagacagccctgggtctcagttttctaagtggcctgcgagtcctgggtcccacagcaacgatgactttgataactggagtacatttcgtcctcgaaccagctcaaatgctagtaccatcagtgggagactttctcccatcatgacagagcaggatgacctgggagatggggacgtgcattccctggtgtatccaccctctgctgccaagatggcgtctacgctgcccagtctgtctgaaatcagcaatccagaaaacatggagaaccttctggataatctcaaccttctctcgtccccaacatctttaactgtgtccacccagtcctcgcctggcagcatgatgcagcagacaccatgctattcgtttgcaccgccaaacaccagtctaaattcacccagtccaaactactcaaagtacacatacggccaatccagcatgagccctttgccccagatgcctatgcagacacttcaggacagcaaatcaagttacggaggattgaaccagtataactgtgccccaggactcttgaaagagttgttgacttctgactctcctccccacaatgacattatgtcaccggttgatcccggagtggcccaacccaacagtcgggtcctgggccaaaatgtaatgatgggccctaattcggtcatgccagcgtatggcagccaggcatctcataacaaaatgatgaaccccagctcccacacccaccctggacatgcacagcaaacggcttcggtcaacggccgtaccctgccccatgtggtgaacaccatgcctcacacatctgccatgaaccgcttgacccccgtgaagacacctttacaagtgcctctgtcccaccccatgcagatgagtgccctgggcagctactcctcggtgagcagctgcaatggctatggtaggatgggtgtcctccaccaggagaagctcccaagtgacttggatggcatgtttattgagcgcttggactgtgacatggagtccatcattcggaatgacctcatggatggagataccttggattttaactttgataatgtgttgcccaaccaaagcttcccacacagtgtcaagactacaacacacagctgggtgtcaggctaagagttagtgagcaggctacatttaaaagtccttcagattgtctgacagcaggaactgaggagcagtccaaagatgcccttcacccctccttatagttttcaagatttaaaaaaaaaaaaaaaaaaaaaaaaagtcctttctcctttcctcagacttggcaacagcggcagcactttcctgtgcaggatgtttgcccagcgtccgcaggttttgtgctcctgtagataaggactgtgccattgggaatcattacaatgaagtgccaaactcactacaccatgtaattgcagaaaagactttcagatcctggagtgctttcaagttttgtatatatgcagtagatacagaattgtatttgtgtgtgtgttttttaatacctacttggtccaaggaaagtttatactcttttgtaatactgtgatggtctcaagtcttgataaactttgctttgtactacctgtgttctgctacagtgagaagtcatgaactaagatctctgtcctgcacctcggctgaatgactgaacctggtcatttgccacagaacccatgagagccaagtagccagtgatcaatgtgctgaattaatggacttgtcaaactttggggcagaataagattaagtgccagctttgtacaggtctttttctattgtttttgttgttgtttattttgttatttgcaaatttgtacaaacaacttaaaatggttctaatttccagataaatgacttttgatgttattgttaggactcaacatcttttggaatagataccgaagtgtaatgttttcttaaaactagagtctactttgttacattgtctgcttataaatttgtgaaatcagaggtatttgggggctgcattcataattttcattttgtatttctaactggattagtactaattttatatgtgctcagctggtttgtacactttgcgatgatacctgataatgtttctgactaatcgtaaaccattgtaattagtacttgcacactcaacgttcctggccctttgggcaggaaagttatgtatagttacagacactctgttttgtgtgtagatttatgtgtgtattttaaagaaatttcacctgcttttattaccctgtgagttgtgtacagcgcatagcaccaagtcttcagatagatgccacgtgcttacagccttctagggaagcctgccagatgatgccctgtgtcacgctgtcatagttcccatgggaactctgtctgtcgctcaggaaaggggaacttttatctaaggtgatgttctttgtctgactggggttcgcctcctactactctgagctgttggcttttgtcacgatggaggtggctttgtggctctgtcctggaagaatcctgtcacttctcggtccccacctctgttctctttggctctgaacagtgtaaatctaaggaggaagtttacaaataggacttcagtgatttatggagtgctctgtgcgcctaagtacagacagtggcaggattagttaaaaatgaaggcagtaaacttggaaaccagccagctataaatggacatttattttgaaatccttagcttaagaatttgagaagttttttcagccttgagcagcctaatgtgtctcaaacatttacgttttttatacattctatttacctgaaatcctgccagaccaggataattggttttacctctcattccgtccatcggtgtttcccagtctcccacagtttgaggaatagatgtaccccagcacccctctttgcctttatgagaaggcctggtttgcatgagaagaccaaattgcacttccatgagaagaccaaattgtttgtagtgttacttagctctcccctcgtttgttagtgtgtgttaacaagaataaaatgtccctgctttcacccaccgttggccagctttgtcataggcttcccaccataactttcactattttaaacacatattgagccactgctcgtctgactacctttgtttgggcactccaaaacaggacttgttttagaaatgaactcctccaagtagagcctccttcaaacagagtagaatttcctggtgtcaaagaacccgggtctgtctccctttcctcctccctctgccatttcttaccattgcggaaagagagagcctccgtgtgtaatcattcagtagaggcagctaccgccctggcagtggtctacctgctgaatgccactgaatgactaggaggtgtctctcccttcagaagctgtcaatttcagcagcaacccctgttttccttggtgttaagatcccagtgtgaatcatgggcagttgtctggggcacagtgaactccaggaaaggcttcgtatctgttttgaaaacaaacatcaaacgtgtgagctccgagggtccttttctgggagaatgttcgctttctggtctattattgtacatgattgctctgtgaaaagacttcatctatgcagccttgtttgattcatttcctttggtgtgttctgttgttaagagcaaattgtattatagagctatttggatattttaaatataaagatgtattgtttccat

SEQ ID NO: 2 - protein, 652 aa, Mus musculus
MAEAPQVVETDPDFEPLPRQRSCTWPLPRPEFNQSNSTTSSPAPSGGAAANPDAAASLASASAVSTDFMSNLSLLEESEDFARAPGCVAVAAAAAASRGLCGDFQGPEAGCVHPAPPQPPPTGPLSQPPPVPPSAAAAAGPLAGQPRKTSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRGRAAKKKASLQSGQEGPGDSPGSQFSKWPASPGSHSNDDFDNWSTFRPRTSSNASTISGRLSPIMTEQDDLGDGDVHSLVYPPSAAKMASTLPSLSEISNPENMENLLDNLNLLSSPTSLTVSTQSSPGSMMQQTPCYSFAPPNTSLNSPSPNYSKYTYGQSSMSPLPQMPMQTLQDSKSSYGGLNQYNCAPGLLKELLTSDSPPHNDIMSPVDPGVAQPNSRVLGQNVMMGPNSVMPAYGSQASHNKMMNPSSHTHPGHAQQTASVNGRTLPHVVNTMPHTSAMNRLTPVKTPLQVPLSHPMQMSALGSYSSVSSCNGYGRMGVLHQEKLPSDLDGMFIERLDCDMESIIRNDLMDGDTLDFNFDNVLPNQSFPHSVKTTTHSWVSG

SEQ ID NO: 3 - DNA, 26 bp, Artificial
  Synthetic Primer
gcttagagcagagatgttctcacatt

SEQ ID NO: 4 - DNA, 26 bp, Artificial
  Synthetic Primer
ccagagtctttgtatcaggcaaataa

SEQ ID NO: 5 - DNA, 24 bp, Artificial
  Synthetic Primer
caagtccattaattcagcacattg

(19) United States
(12) Patent Application Publication
(10) Pub. No.: US 2008/0260700 A1
(43) Pub. Date: Oct. 23, 2008

(54) GENERATION OF PANCREATIC ENDOCRINE CELLS FROM PRIMARY DUCT CELL CULTURES AND METHODS OF USE FOR TREATMENT OF DIABETES

(75) Inventors: Domenico ACCILI, New York, NY (US); Tadahiro Kitamura, Fort Lee, NJ (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/037,401

(22) Filed: Feb. 26, 2008

Related U.S. Application Data
(63) Continuation-in-part of application No. PCT/US06/33419, filed on Aug. 28, 2006.
(60) Provisional application No. 60/711,591, filed on Aug. 26, 2005.

(51) Int. Cl. A61K 35/12; C12N 5/00; A61P 3/10; C12N 5/08

(52) U.S. Cl. 424/93.7; 435/325; 435/366

(57) ABSTRACT

The invention is directed to spontaneously immortalized pancreatic duct cells and methods for generating pancreatic endocrine cells from spontaneously immortalized pancreatic duct cells that express the transcription factors Pdx1 and FoxO1. The invention also provides for methods for treating beta cell failure, the method comprising administering to a subject an effective amount of spontaneously immortalized pancreatic duct cells expressing a mutated version FoxO1.

This application is a continuation-in-part of International Application No. PCT/US2006/033419, filed Aug. 28, 2006, and claims priority to U.S. Provisional Application No. 60/711,591, filed Aug. 26, 2005; both applications are herein incorporated by reference in their entireties.

The invention disclosed herein was made with U.S. Government support under NIH Grant No. 1R01DK64618 from the NIDDK. Accordingly, the U.S. Government may have certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND

Diabetes is the result of impaired insulin production from pancreatic endocrine beta-cells. Pancreatic duct cells are considered to be progenitors of pancreatic endocrine cells in adult pancreas. The mechanism of pancreatic duct cell differentiation into endocrine cells is unclear.

Pancreatic duct cell lines are useful tools for the study of duct cell differentiation into endocrine cells. Existing pancreatic duct cell lines as described in the art are derived from pancreatic cancers are not suitable for differentiation studies. Conventional approaches to isolate primary pancreatic duct cells from rodents have been described (Arkle et al., 1986). These approaches used micropuncture methods on isolated duct structures from rats. However, intralobular ducts, or ductules, are too small to be collected by conventional manual approaches. Another problem in the art related to isolating and purifying duct cells from pancreas is that, because small ducts are associated with acinar tissue, small vessels and connective tissue, it is difficult to exclude these associated components from the duct cells. Finally, it is virtually impossible to exclude contamination of retrieved duct cells with endocrine cells.

It is important to better define pancreatic duct cell differentiation and to establish methods to produce hormone-producing endocrine cells from duct cells, because they may play an important role in the development of new treatments for diabetes.

### SUMMARY

The invention provides for an immortalized pancreatic duct cell derived from a primary adult pancreatic duct epithelial cell that is capable of acquiring features of an endocrine cell. In another aspect, the invention provides for an immortalized pancreatic duct cell derived from a primary adult pancreatic duct epithelial cell, wherein the cell expresses a mutated version of the DNA transcription factor Fox01, which is constitutively retained in the cell nucleus, unlike the wild-type Fox01, which shuttles between the nucleus and the cytoplasm. In another aspect, the invention provides for a cell derived from a primary pancreatic duct cell (for example, using the method of the invention) where the cell is capable of producing pancreatic hormones. In one embodiment, the duct-derived cell contains an exogenous nucleic acid encoding a mutated Fox01; the mutation abolishes the ability of Fox01 to become phosphorylated. In one embodiment, the mutation comprises substitution of serine 253 of SEQ ID NO:2 with alanine. The invention also provides a method for treating pancreatic beta cell failure, the method comprising administering to a subject in need thereof an effective amount of pancreatic ductal cells that express mutated Fox01.

A spontaneously immortalized pancreatic duct cell line of the invention, designated 24-1 Duct, was deposited pursuant to the Budapest Treaty on the International Recognition of the Deposit of Microorganisms for the Purposes of a Patent Procedure with the Patent Depository of the American Type Culture Collection (ATCC), 10801 University Blvd., Manassas, Va., 20110, on Aug. 25, 2005, and accorded ATCC Accession No. PTA-6968.

In one aspect, the invention provides for an immortalized pancreatic duct cell derived from a primary adult pancreatic duct epithelial cell culture, wherein the immortalized cell expresses Pdx1 and/or Fox01. (Throughout this application, the use of the conjunction “and” includes within it, the conjunction “or”, unless otherwise specified.) In another embodiment, the cell lines expresses pancreatic duct cell markers. For example, the pancreatic duct cell markers comprise cytokeratin 16 and/or carbonic anhydrase II. In another embodiment, the cells of the cell line of the invention do not express endocrine pancreatic markers. For example, the endocrine pancreatic markers comprise insulin, glucagon, somatostatin and/or pancreatic polypeptide. In another aspect, the cell does not express exocrine pancreatic markers. In another embodiment, the exocrine pancreatic markers comprise amylase, trypsin and/or elastase.

In accordance with aspects of this invention, the pancreatic duct cells and cells derived therefrom may comprise a cell line designated 24-1 Duct and having ATCC Accession No. PTA-6968, and deposited on Aug. 25, 2005 with the Patent Depository of the ATCC, 10801 University Blvd., Manassas, Va., 20110, under the provisions of the Budapest Treaty on the International Recognition of the Deposit of Microorganisms for the Purposes of a Patent Procedure.

In one aspect, the invention provides for a human pancreatic duct cell line. In another aspect, the invention provides for an immortalized pancreatic duct cell derived from a primary adult pancreatic duct epithelial cell, wherein the cell expresses mutated Fox01, wherein the mutation causes Fox01 to lose its ability to be phosphorylated. For, example mutation of serine 253 of SEQ ID NO:2 to alanine. In one aspect of the invention, the mutated version of Fox01 contains a loss-of-function mutation. In one embodiment, the mutation of Fox01 comprises a mutation that results in a truncation of the transactivation domain of the Fox01 protein. In one embodiment, the last 400 amino acids (or approximately 400 amino acids) of the Fox01 protein (SEQ ID NO:2) are truncated. This truncation renders Fox01 inactive, because it removes the so-called “transactivation domain” which is required to transcribe DNA into RNA. The coding sequence of the murine homologue of FoxO1 is represented by nucleotides 429-2387 of SEQ ID NO:1. The invention also provides for expression of mutated versions of the human homologue of Fox01 (the nucleotide sequence encoding human Foxo01 is shown for example in GenBank Accession No. NP_{—}002006).

In another aspect of the invention, the cells of the cell line express endocrine pancreatic markers. For example, the endocrine pancreatic markers can comprise Isl1, Nkx6.1, Nkx2.2, NeurodD1, glucagon and/or pancreatic polypeptide.

The invention provides for a method for obtaining a pancreatic duct cell line, the method comprising: (a) culturing pancreatic duct cells collected from a subject in medium comprising about 10% serum and about 5.5 mM glucose for about a week; (b) culturing the cells in a medium comprising (i) about 8 mM glucose; (ii) about 1 g/L ITS (about 5 mg/l insulin+5 mg/l transferrin+5 mg/l selectin), (iii) about 2 g/l albumin, (iv) about 10 mM nicotinamide, and (v) about 10 mg/ml keratinocyte growth factor, for about at least another week until the culture comprises nearly all duct cells; (c) culturing the duct cells with the medium of step (b) further comprising about 10% serum and about 5.5 mM glucose; (d) passaging the cells of step (c) until the cells' doubling time reach about 24 hours; and (e) cloning a single cell from the cells of step (d) so as to obtain a clonal pancreatic duct cell line.

The invention provides for a method for producing a pancreatic hormone-producing cell, the method comprising culturing immortalized pancreatic duct epithelial cells under the conditions described herein. This invention provides for a method to isolate cells from pancreas duct with a potential to become hormone-producing cells, including insulin-producing beta cell conditions, wherein the immortalized pancreatic duct cells express mutated Fox01. In one embodiment, the Fox01 mutant comprises a mutation of serine 253 to alanine.

The invention provides for a method for treating beta cell failure, the method comprising administering to a subject one or more cells from a spontaneously immortalized pancreatic ductal cell line expressing mutated Fox01. In one embodiment, the cell line is the cell line that was deposited with the ATCC on Aug. 25, 2005 under the provisions of the Budapest Treaty, designated 24-1 Duct and having ATCC Accession No. PTA-6968. In another embodiment, the administering comprises transplanting a sponge matrix comprising immortalized pancreatic ductal cells expressing mutated Fox01. In another embodiment, the invention provides for administering to the subject cells that are capable of producing pancreatic hormones that are derived from a pancreatic duct cell line of the invention.

The subject on which the method is employed may be any mammal, e.g. a human, mouse, cow, pig, dog, cat, or monkey. In one embodiment, the administering comprises infusion, injection, incapsulation, or any combination thereof. The administration of the cells may be effected by intralesional, intraperitoneal, intramuscular or intravenous injection; by infusion; or may involve surgical implantation, carrier-mediated delivery; or topical, nasal, oral, anal, ocular or otic delivery.

In the practice of the method, administration of the inhibitor may comprise daily, weekly, monthly or hourly administration, the precise frequency being subject to various variables such as age and condition of the subject, amount to be administered, half-life of the agent in the subject, area of the subject to which administration is desired and the like.

In connection with the method of this invention, a therapeutically effective amount of the cells may include dosages which take into account the size and weight of the subject, the age of the subject, the severity of the beta cell failure, the method of delivery of the cells and the history of the symptoms in the subject.

### BRIEF DESCRIPTION OF THE FIGURES

FIGS. 1A-1B. Primary pancreatic duct cell culture. After isolation of the cells from murine pancreatic tissue as described in Example 1, the cells were cultured for 7 days in medium containing serum. Cobblestone-like duct cells and spindle-shaped fibroblast-like cells were observed in the culture (FIG. 1A). Serum-containing medium was then replaced with serum-free medium. After 7 days of culture in serum-free medium, the fibroblast-like cells stopped growing and detached from the culture dishes, while the duct cells were still proliferating even after 14 days in serum-free medium (FIG. 1B).

FIGS. 2A-2B. Primary pancreatic duct cell culture (following 2 weeks in culture in serum-free medium). After 14 days in serum-free medium, cultured cells were subjected to immunostaining with anti-pancytokeratin antibody to identify cytokeratin-positive pancreatic duct epithelial cells in the culture.

FIGS. 3A-3D. Pancreatic duct cell culture (following 8 weeks culture in serum-free medium). After 8 weeks of culture in serum-free medium, only duct cells remained in the culture. This observation was confirmed by immunostaining with anti-pancytokeratin antibody (FIGS. 3A-3B) and anti-Pdx1 antibody (FIGS. 3C-3D).

FIGS. 4A-4F. Pancytokeratin and vimentin expression in single-cell cloned primary pancreatic duct cell culture. Single cell cloning was conducted by the limiting dilution method and 24 independent cell lines were obtained. Based on morphological observations, the cell lines were classified into two groups: Clone i) consisted of purely cobblestone-like cell lines which expressed cytokeratin (epithelial marker) but not vimentin (mesenchymal marker) (FIGS. 4A-4C, top row); and Clone ii) consisted of spindle-like cell lines which expressed both cytokeratin and vimentin (FIGS. 4D-4F, bottom row).

FIG. 5. Karyotype of primary pancreatic duct cell culture. Chromosomal analysis (karyotype) was performed on five representative cell lines. All cell lines analyzed had abnormal chromosomes compared to the normal mouse chromosome (N=42). Four of the five cell lines analyzed contained 74 chromosomes and one of the five cell lines analyzed contained 44 chromosomes, indicating that these cell lines were spontaneously transformed.

FIGS. 6A-6D. Complete mis-localization of Fox01 with Pdx1 in primary pancreatic duct cell culture. Spontaneously immortalized pancreatic duct cells express two important transcription factors for endocrine cell differentiation. Immunostaining results show the expression of Pdx1 and Fox01.

FIGS. 7A-7F. Glucagon and pancreatic polypeptide are induced by Fox01 in primary duct cell culture. A mutant version of the transcription factor Fox01 was introduced into the cultured duct cells (mutation of serine 253 of SEQ ID NO:2 to alanine). After a week in culture, duct cells expressing the mutant Fox01 were positive for glucagon and pancreatic polypeptide, as determined by immunohistochemistry. The top row shows cells transduced with a control virus. The bottom row shows cells transduced with the mutant Fox01 virus. The left column shows staining with antiserum against glucagon, a hormone. The staining indicates that glucagon production is present in cells expressing Fox01, but not in cells transduced with the control virus. The middle column shows staining for another hormone called pancreatic polypeptide. The staining in this case indicates that there is production of pancreatic polypeptide. Again, it is only seen in cells expressing Fox01. The right column shows co-staining for glucagon and DNA (DAPI). This is done to mark individual cells and determine how many cells express glucagon. Virtually all cells transduced with Fox01 express glucagon. This is a notable finding, because thus far there have been no methods in which all cells would become (i.e., differentiate into) hormone-producing cells.

FIGS. 8A-8B. Nucleotide sequence of mouse (Mus musculus) forkhead box O1 (Foxo1) (GenBank Accession No. NM_{—}019739; SEQ ID NO:1)

FIG. 9. The amino acid sequence of mouse (Mus musculus) forkhead box O1 (Foxo1) encoded by nucleotides 429-2387 of SEQ ID NO:1 shown in FIGS. 8A-8B (GenBank Accession No. NP_{—}062713; SEQ ID NO:2).

FIGS. 10A-10L. FoxO1 localization in adult mouse pancreatic islets. Pancreatic sections from 2 month-old mice were analyzed by immunohistochemistry with antibodies against FoxO1 (B-C, E-F, H-I, K-L), glucagon (A, C), pancreatic polypeptide (D, F), somatostatin (G, I), or insulin (J, L).

FIGS. 11A-11I. FoxO1+ cells in pancreatic ducts. Pancreatic immunohistochemistry with anti-Fox01 (A, D, G) and anti-insulin (B, E, H) antibodies. (A-F) All insulin+ juxtaductal cells co-express FoxO1. (D-I) FoxO1+Ins− cells are located near to (D-F) or within ducts (G-I).

FIGS. 12A-12O. Developmental analysis of FoxO1 expression in embryonic pancreas. Pancreatic sections from Neurog3-Gfp transgenic (A-C) and wildtype mice (D-O) at E14.5, E17.5, and post-natal day 28 (P28) were analyzed by double immunohistochemistry with antibodies against Gfp and amylase (A-C), or by histochemistry with DBA (D-F), or immunohistochemistry with antibodies against FoxO1 (G-I), Pdx1 (J-L), and Foxa2 (M-O).

FIGS. 13A-13R. Sub-cellular localization of FoxO1 in different pancreatic compartments at E17.5. Immunostaining with anti-FoxO1 (A, C, D, F, G, I, J, L, M, O, P, R) and anti-amylase (B-C), DBA (E-F), Cytokeratin (H-I), Gfp (Neurog3) (K-L), Glucagon (N-O) or insulin (Q-R).

FIGS. 14A-14S. Abnormal pancreas development in Pdx-FoxO1ADA transgenic mice. (AB) FLAG immunohistochemistry in E7.5 transgenic (A) and control mice (B). Brown arrows denote budding acini that are entirely FLAG+; the blue arrow indicates a FLAG-cell. (C-D) Photomicrograph of the gastroduodenal tract in transgenic (C) and control mice (D). (E-H) H&E staining of pancreatic sections from transgenic and control mice at 40× (E-F) or 200× magnification (G-H). Remnants of the exocrine pancreas are marked by the dashed line. (I-J) FLAG immunohistochemistry in 3 month-old transgenic (I) and control mice (J). Brown arrows denote a large FLAG+ islet; blue arrows indicate FLAG-islets. (K-N) Insulin and (O-R) glucagon immunohistochemistry in transgenic (K, M, O and Q) and control mice (L, N, P and R) at 40× (K-L and O-P) or 200× magnification (M-N and Q-R). (S) Ratio of islet b to a cells in control (WT) and Pdx-Foxo1ADA (TG) mice. An asterisk indicates P<0.01 by ANOVA.

FIGS. 15A-15H. Conditional inactivation and ductal immunohistochemistry in mice homozygous for Fox01 conditional null alleles. (A) Genotyping of DNA isolated from whole pancreas, duodenum or liver of Hs1(cre):FoxO1^{-/-} (lanes 1), Pdx1(cre):FoxO1^{-/-} (lanes 2), Neurog3(cre):FoxO1^{-/-} (lanes 3), Ins(cre):FoxO1^{-/-} (lanes 4), FoxO1^{flox/flox} (lanes 5) for multiplex detection of WT, floxed and deleted (ko) alleles (upper panel) or for single detection of the deleted allele (ko) (lower panel). Hs1(cre) transgenic mice are an embryonic deleter strain used as positive control for recombination (Dietrich et al., 2000). (B) Immunohistochemistry of representative pancreatic sections from Pdx1 (cre):FoxO1^{-/-} (upper panel) and FoxO1^{flox/flox} mice (lower panel). (C) Immunohistochemistry of representative sections from Pdx1(cre):FoxO1^{-/-} mice with DAPI, anti-Pdx1, anti-Ki67 and anti-insulin antibodies. Images are shown at 40× magnification. (D) Ki67 labeling index of juxta-ductal (empty bar) or islet β cells (full bar) in Pdx1(cre):FoxO1^{-/-} mice. An asterisk indicates P<0.01 by ANOVA. (E-H) Double immunohistochemistry with anti-insulin and anticytokeratin (E-F), anti-glucagon (G), or anti-Nkx2.2 antibodies (H). Images are shown at 10× (E) or 100× magnification (F-H).

FIGS. 16A-16C. Establishment of primary pancreatic cell cultures. (A) Immunocytochemistry with anti-pancytokeratin, anti-vimentin antibodies and DAPI shown at 10× magnification. (B) After single cell cloning by limiting dilution, each clone was incubated with X-Gal to detect β-galactosidase activity. Representative β-gal+ and β-gal− clones are shown. (C) Immunocytochemistry of a representative clone with anti-Fox01, anti-Pdx1 antibodies and DAPI. Images are shown at 40× magnification.

FIGS. 17A-17D. Endocrine-like differentiation of FoxO+Ins− cells. (A) mRNA expression analysis of a representative clone of FoxO+Ins− cells transduced with adenovirus expressing constitutively active FoxO1ADA or GFP. mRNA was amplified by RT-PCR. In control samples, the reverse transcriptase step was omitted (“−” sign). Where possible, mRNA isolated from αTC3 or βTC3 cells was used as positive control. (B) Glucagon immunocytochemistry in cells transduced with adenovirus encoding FoxO1ADA or GFP. (C) Clone #1 and control cells, including embryonic ureteric bud (UB) cells, kidney cortical collecting duct cells (M-1), pancreatic acinar carcinoma cells (TGP47) and SV40-transformed hepatocytes, were transduced with FoxO1 ADA adenovirus. After isolating mRNA, semi-quantitative RT-PCR was performed with primers for glucagons. (D) Expression of Pdx1, NeuroD, Ins1, Ins2 and Gluc in clone #1, following transfection of Fox01 or control siRNA.

### DETAILED DESCRIPTION

The issued patents, applications, and other publications that are cited herein are hereby incorporated by reference to the same extent as if each was specifically and individually indicated to be incorporated by reference.

Insulin-producing β cells are central to the pathogenesis of diabetes. Understanding the mechanisms governing their ontogeny may offer strategies for their somatic replacement. Previous efforts to obtain functional β cells from differentiation of embryo-derived stem cells or from cells committed to the pancreatic lineage, including duct epithelial cells, have met with limited success. As disclosed herein conditional FoxO1 ablation in pancreatic progenitor cells, but not in committed endocrine progenitors or terminally differentiated β cells, results in a selective increase of juxta-ductal β cells that are phenotypically distinct from duct epithelial cells. Multiple clonal isolates and derivative permanent cultures of these FoxO1+ cells were established and assayed for their capacity to undergo endocrine differentiation in vitro. The FoxO1+ cultures were able to convert into endocrine-like, glucagon-producing cells. FoxO1+ juxta-ductal cells represent a hitherto unrecognized pancreatic cell population with limited in vitro capability of endocrine differentiation.

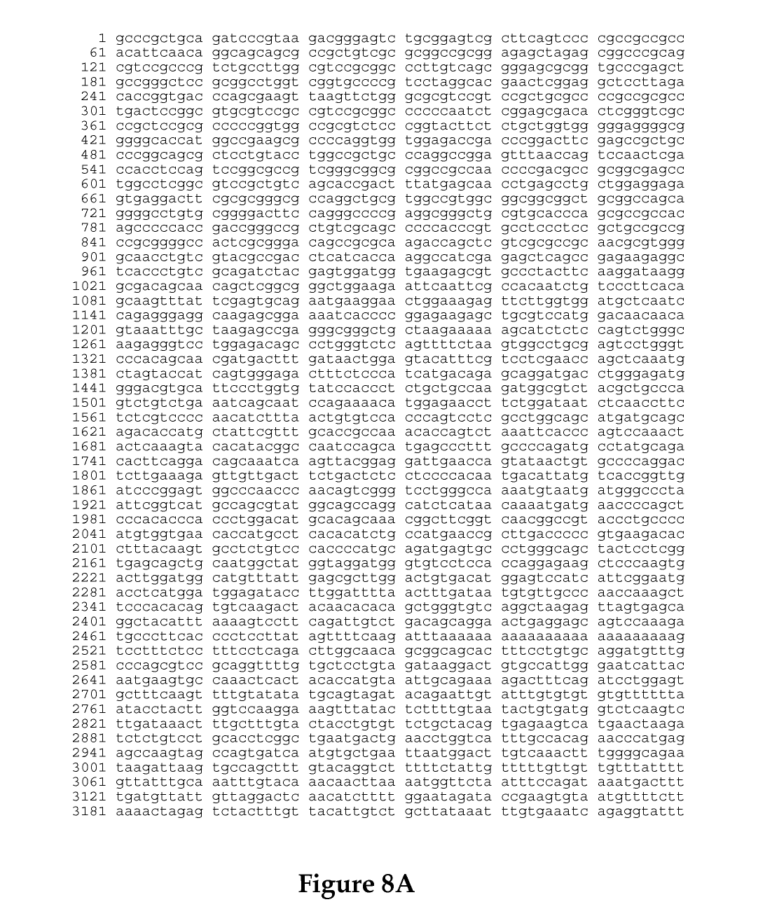

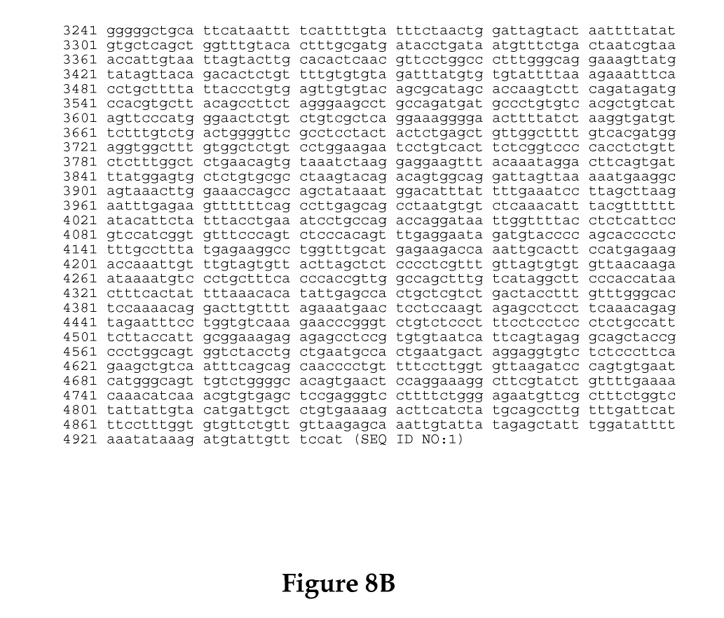

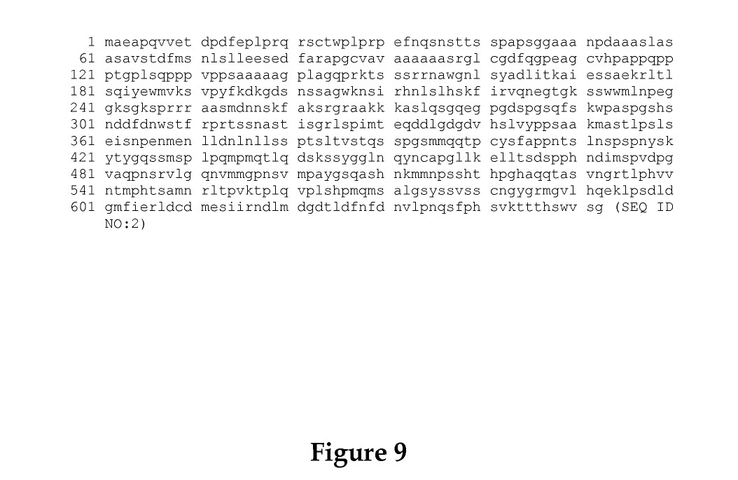

It is a discovery of the present invention that a select population of endocrine progenitor cells is located in pancreatic ducts. This population of pancreatic duct cells can give rise to hormone-secreting pancreatic endocrine cells. These pancreatic duct cells are characterized by expression of the transcription factors Pdx1 and Fox01. The nucleotide sequence and amino acid sequence of FoxO1 are shown as SEQ ID NO:1 (FIGS. 8A-8B) and SEQ ID NO:2 (FIG. 9), respectively. Proliferation and differentiation of pancreatic endocrine cells is regulated by the expression of Pdx1 in the nucleus. Fox01 expression in the nucleus acts as a negative regulator of endocrine cell proliferation and differentiation by decreasing the expression of Pdx1. Upon translocation of Fox01 from the nucleus to the cytoplasm, the expression of Pdx1 in the nucleus increases, thus enhancing the proliferation and differentiation of pancreatic endocrine cells.

The cells of the invention were obtained from primary cultures of pancreatic ducts. Currently, there are no permanent cell lines derived from normal pancreatic ducts. There are two cell lines derived from pancreatic carcinomas, which generally arise from pancreatic ducts. Such cell lines are generally referred to as “pancreatic ductal” cells, but they are highly abnormal and do not express most of the markers found to be expressed by a normal pancreatic duct cell.

Cells of the invention were obtained by first removing pancreatic acinar cells through filtration, then removing pancreatic islet cells by centrifugation. The supernatant of the centrifugation was plated on a gelatin-coated culture dish and cells were allowed to replicate. Most cells died within two weeks of being plated, however, those that survived underwent spontaneous immortalization. The surviving cells were isolated and cloned by limiting dilution. This process has been applied to many other cell types, but never before to pancreatic duct cells. Through this process, individual cells were isolated. The isolation of single-cell (“clonal”) populations is also a discovery of the invention, as no other clonal cell lines have been derived from pancreatic ducts. The lineage (derivation) of the cells has been confirmed by measuring the expression of ˜40 different genes that are typical of ductal epithelial cells. They include Pdx1, Cytokeratin 16, 18, 20, vimentin, Carbonic anhydrase II and many others. Other genes whose expression is characteristic of ductal epithelial cells include: Glucagon, Pancreatic Polypeptide, Amylase, Pdx1, Nkx2.2, Nkx6.1, Pax6, NeuroD, Ptf1(p48), MafA, Ck19, Carbonic Anhydrase2, Vimentin, Foxa2, Hes1, CBF₁, Notch1, Sirt1, AFP, and PML. The expression of genes that are not expressed in ductal cells was also measured, such as insulin, glucagon, pancreatic polypeptide, amylase, Somatostatin, Neurogenin3, Brain4, Arx, Elastase, and/or Trypsin. Techniques for measuring gene expression are known in the art. Non-limiting examples include in situ hybridization, PCR-based methods and microarray analysis.

The invention provides for an immortalized pancreatic duct cell derived from a primary adult pancreatic duct epithelial cell culture, wherein the immortalized cell expresses Pdx1 and Fox01. The invention provides for methods to obtain/produce hormone-producing pancreatic endocrine cells. Such cells would be useful in the treatment of diseases, such as type 1 and type 2 diabetes. In this invention, a method is provided for converting pancreatic duct cells into hormone producing cells by way of a specific genetic alteration. In one embodiment, the genetic alteration comprises silencing expression of Fox01 via RNA interference (RNAi), or by introducing dominant-negative mutants that inhibit the action of the endogenous Fox01 gene. In one embodiment, the cells have been obtained from primary cultures of pancreatic ducts. Currently, there are no permanent cell lines available that are derived from normal pancreatic ducts. The cell lines derived from pancreatic carcinoma arise from pancreatic ducts. These cells are generally referred to in the literature as “pancreatic ductal”, but they are highly abnormal and do not express most of the markers of a normal pancreatic duct. This invention provides for cells that have been obtained by removing first pancreatic acinar cells through filtration, then removing pancreatic islet cells by centrifugation. The supernatant of the centrifugation is then been plated on a gelatin-coated culture dish and cells are allowed to replicate. Most cells die within two weeks of being plated, but those that survive have undergone spontaneous immortalization. The surviving cells are isolated and cloned by limiting dilution. This process has not before been applied to pancreatic ductal cells. Then individual cells were isolated. The isolation of single-cell (“clonal”) populations is an advancement of this invention, since no other clonal cell lines have been derived from pancreatic ducts prior to this invention. The lineage (derivation) of the cells has been confirmed by measuring the expression of ˜40 different genes that are typical of ductal epithelial cells. They include Pdx1, Cytokeratin 16, 18, 20, vimentin, Carbonic anhydrase II and many others as explained below. In addition, the expression of genes that should not be expressed in duct cells, such as insulin, glucagon, pancreatic polypeptide and amylase were also measured.

The mutation of Fox01 abolished phosphorylation of Fox01 and caused the protein to localize constitutively to the nucleus of the cell. In one embodiment, the serine at amino acid position 253 of SEQ ID NO:2 is replaced by alanine, a non-phosphorylatable amino acid. Techniques and kits for mutating amino acids and expression of mutated proteins are known in the art (for example, the QuikChange® Site-Directed Mutagenesis Kit (Stratagene)). Normally Fox01 protein shuttles between the nucleus and the cytoplasm. The Fox01 mutant protein was introduced by adenoviral-mediated transduction. Transduction of the Fox01 mutant in other types of viral vectors would be apparent to one skilled in the art.

The present invention provides for methods to isolate, select and culture pancreatic duct cells. In one embodiment of the invention, isolated primary duct cells were cultured for at least 10 months and were spontaneously immortalized. Single cell cloning was performed using limiting dilution methods and 24 cell lines resulted. The cell lines retained duct epithelial characteristics, for example, cytokeratin, carbonic anhydrase II and lectin expression. The cell lines also express Pdx1, an important transcription factor for pancreatic endocrine cell differentiation. The results shown for this invention indicate that the immortalized pancreatic duct cells can function as pancreatic endocrine precursors.

An immortalized pancreatic duct cell line of the invention, designated 24-1 Duct and having ATCC Accession No. PTA-6968, was deposited with the Patent Depository of the American Type Culture Collection (ATCC), 10801 University Blvd., Manassas, Va., 20110, on Aug. 25, 2005, under the provisions of the Budapest Treaty on the International Recognition of the Deposit of Microorganisms for the Purposes of a Patent Procedure.

The invention provides methods to culture cells from pancreatic ducts obtained from a mammal (such as a human, or a mouse) and convert them into hormone-producing endocrine cells using adenoviral gene transfer.

The following nonstandard abbreviations used herein: Neurog3: Neurogenin-3; Pdx1: pancreas and duodenum homeobox protein-1; Amy: amylase; Cytokeratin: Ck; Vimentin: Vm; Foxa2: forkhead box-containing protein A2; Pml; promyelocytic leukemia-associated protein; Nkx: homeodomain protein; Carbonic anhydrase: Ca; Elastase: Ela; Trypsin: Try; Pancreatic polypeptide: Pp; Somatostatin: Ssn; Glucagon: Gluc; NeuroD: neural differentiation-associated transcription factor D2; Pax: paired-box gene; Arx: homeobox containing gene on chromosome X; Bm4: POU homeodomain protein brain-4; Ptf1: pancreas transcription factor-1; Maf: v-Maf cellular ortholog bZIP protein.

The term “carrier” is used herein to refer to a pharmaceutically acceptable vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of ≦20%.

The term “effective” is used herein to indicate that the inhibitor is administered in an amount and at an interval that results in the desired treatment or improvement in the disorder or condition being treated (e.g., an amount effective to reduce body weight of a subject, or to reduce insulin resistance).

In some embodiments, the subject is a mammal. Nonlimiting examples of mammals include: human, primate, mouse, otter, rat, and dog.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

The following examples illustrate the present invention, and are set forth to aid in the understanding of the invention, and should not be construed to limit in any way the scope of the invention as defined in the claims which follow thereafter.

### EXAMPLES

### Example 1

### Isolation, Culture, and Immortalization of Murine Pancreatic Duct Cells

Pancreatic duct cells are considered to be progenitors of pancreatic endocrine cells in adult pancreas. The clarification of pancreatic duct cell differentiation and the establishment of the methods to produce hormone-secreting cells from duct cells can contribute to the development of new treatments for diabetes.

Pancreatic duct cell lines are useful tools for the study of duct cell differentiation. Existing pancreatic duct cell lines are derived form pancreatic cancers and are not suitable for differentiation studies. Conventional approaches to isolate primary pancreatic duct cells from rodent have been described (Arkle et al., 1986). These approaches used the micropuncture method on isolated duct structure from rats. However, intralobular ducts (ductules) are too small to be collected by these manual approaches. Another problem for isolating and purifying duct cells from pancreas is that because small ducts are basically associated with acinar tissue, small vessels and connective tissue, it is considerably difficult to exclude these associated components from duct cells.

The invention provides for newly established methods to isolate, select and culture the pancreatic duct cells efficiently from mice and other mammals, such as humans. Duct cells obtained using the methods of the invention have been cultured for at least 10 months and were confirmed to be spontaneously immortalized. Single cell cloning was performed using limiting dilution methods and obtained 24 cell lines. Some of these cell lines retained duct epithelial characteristics (cytokeratin, carbonic anhydrase II and lectin expression). These cell lines express Pdx1, which is an important transcription factor for pancreatic endocrine cell differentiation, suggesting that the cells have the potential to function as pancreatic endocrine progenitors.

Isolation of pancreatic duct components from 2-month-old C57BL/6J mice: Mice were anesthetized using pentobarbital sodium. A midline abdominal incision was made and 3 ml of M199 medium containing 1 mg/ml collagenase P (Roche Molecular Biochemicals) was injected into the common bile duct, then the swollen pancreas was removed and incubated them at 37° C. for 17 min. Thereafter, 30 ml of ice-cold M199 medium containing 10% newborn calf serum (NCS) was added to stop the digestion reaction. The digested pancreas was dispersed with 30 ml of the same medium. After rinsing with the same medium twice, the tissue suspension was applied to a Spectra-mesh (408 μm; Spectrum Laboratories, Inc) to remove the through-passed small components including acinar tissue and islets, then collected the remaining tissue on the mesh. Thereafter, the collected tissue was resuspended in RPMI medium containing 10% FCS and 5.5 mM glucose and cultured them at 37° C. in 5% CO₂.

One week later, cobblestone-like cells (typical morphology for duct structure) were observed along with the spindle-like cells (typical for fibroblast structure) in the cell culture. Culture was begun in serum-free RPMI medium supplemented with 8 mM glucose, 1 g/l ITS (5 mg/l insulin+5 mg/l transferring+5 mg/l selectin, Sigma), 2 g/l BSA, 10 mM nicotinamide (Sigma) and 10 ng/ml keratinocyte growth factor (KGF). From this time on, the serum-free medium was replaced every 4 days.

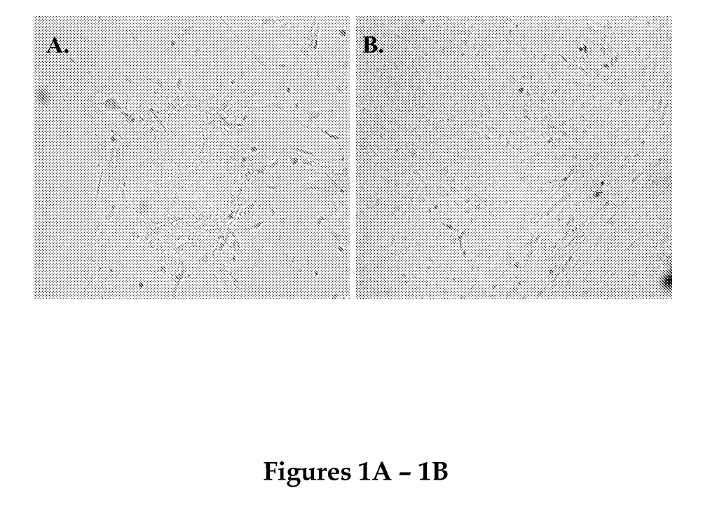

After 7 days of culture in the serum-free medium, fibroblast-like cells stopped growing and detached from culture dishes, while cobblestone-like cells (presumably duct cells) were still proliferating even in serum-free medium (FIG. 1).

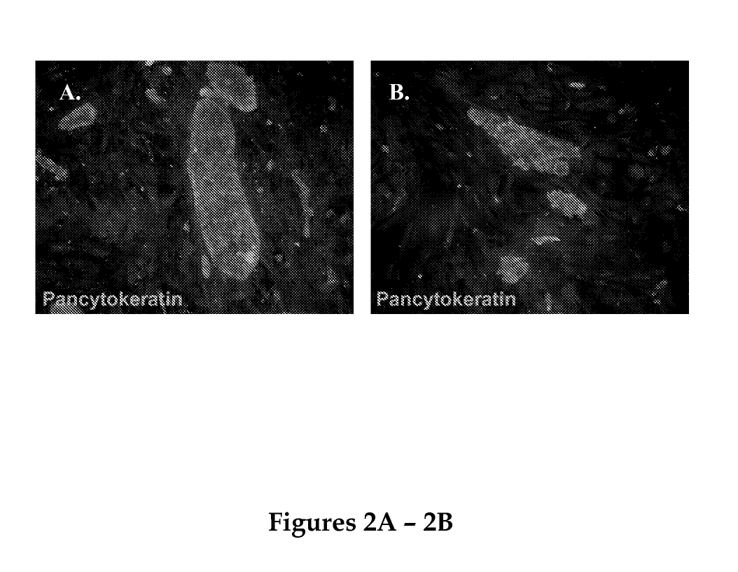

After another week, the cell culture was immunostained with anti-pancytokeratin antibody (Sigma) and identified cytokeratin-positive duct epithelial cells in the culture (FIG. 2).

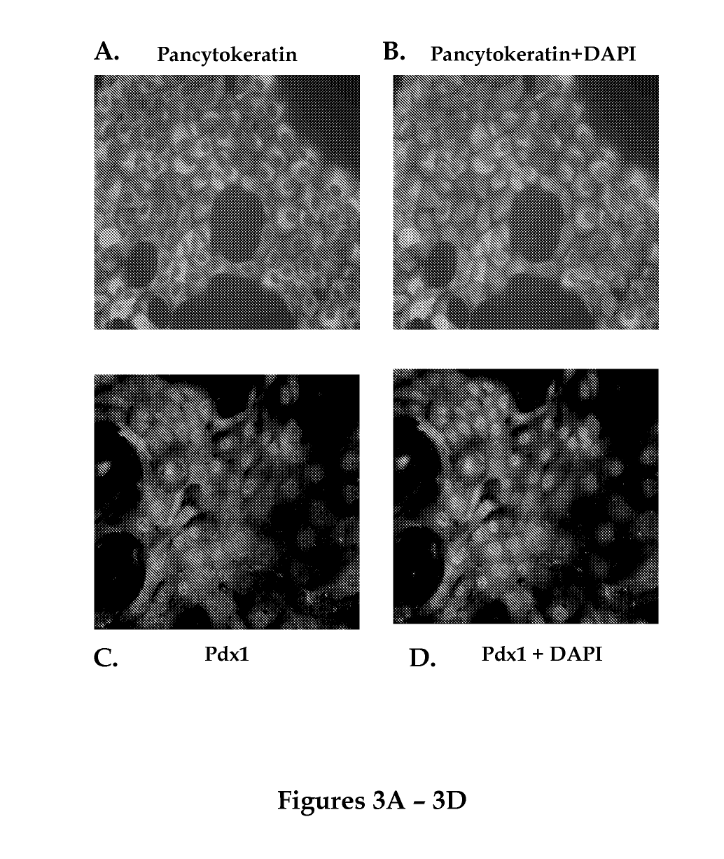

After 8 weeks of culture in serum-free conditions, only duct cells remained in the culture. This was confirmed by immunostaining with anti-pancytokeratin antibody (FIG. 3 upper panel). Cells were attached to culture dishes and looked healthy, but their growth stopped, indicating that they were in a senescent phase. Therefore, fetal calf serum (FCS) was supplied in the culture medium again. The medium supplemented with 10% FCS and 5.5 mM glucose was refreshed every 4 days.

After 2 weeks of culture in serum-containing medium, cells resumed to grow. The doubling time was 72 hrs. The cells were passaged every 7 days at a ratio of 1:3 using 0.05% Trypsin/0.02% EDTA.

The proliferation rate of the cells gradually increased. After ten passages, the doubling time achieved to 24 hrs. The cells were passaged every 3 days at a dilution of 1:5.

To get a homogeneous population of the cells, single cell cloning was conducted using the limiting dilution method.

As a result of single cell cloning, 24 independent cell lines were obtained. From morphological observations, the cell lines were classified into two groups: i) purely cobblestone-like cell lines, and ii) spindle-like cell lines. The growth rate was different between two groups. The doubling time of group i) and ii) were 36 hrs and 18 hrs, respectively.

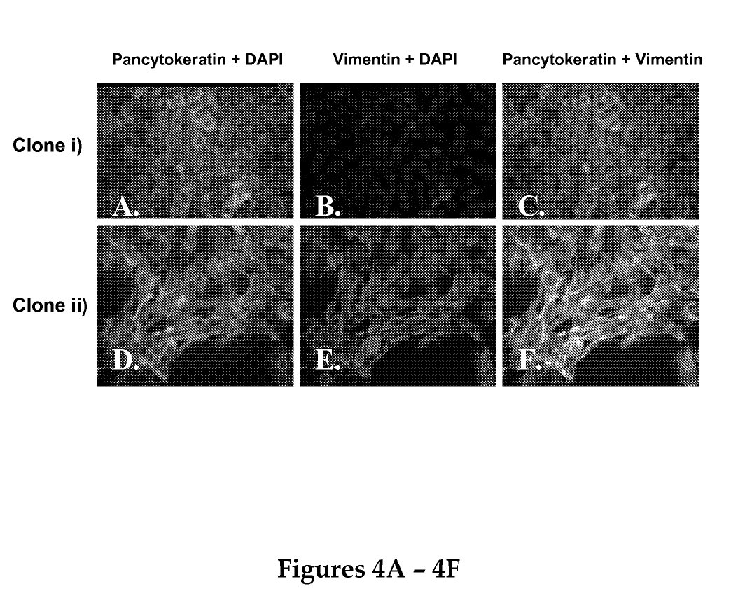

Immunostaining was performed to characterize the cells. The cells in group i) expressed cytokeratin (epithelial marker) but not vimentin (mesenchymal marker), while the cells in group ii) expressed both cytokeratin and vimentin. Each clonal cell line was passaged every 3 days at a ratio of 1:3˜1:5. This stable rate of cell growth was maintained through at least 50 passages, indicating that the cells had been immortalized (FIG. 4).

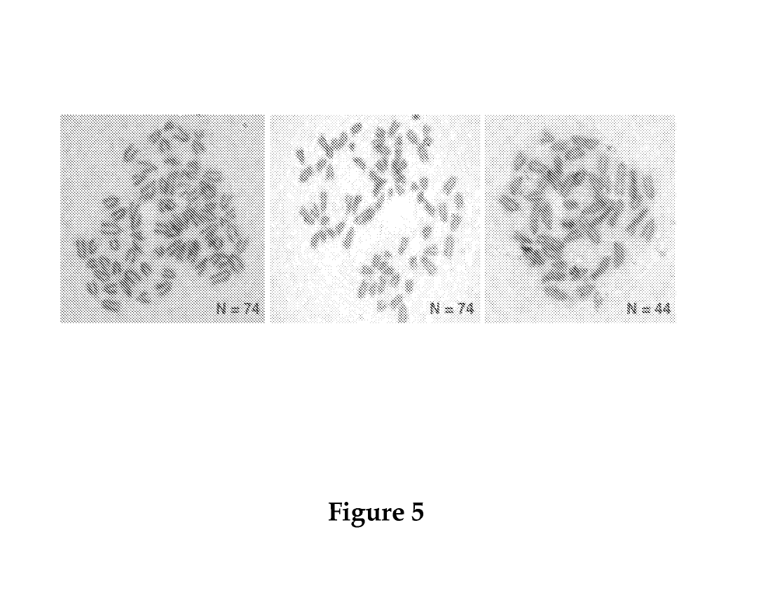

Chromosomal analysis (karyotype) was performed on five representative cell lines. As expected, all cell lines had abnormal chromosomes compared to normal mouse chromosomes (N=42). Four of the five cells lines contained 74 chromosomes and one of the five cell lines contained 44 chromosomes (FIG. 5), indicating that these cell lines were spontaneously transformed. These cell lines were named Spontaneously Immortalized Pancreatic Duct Cells (SIPDC).

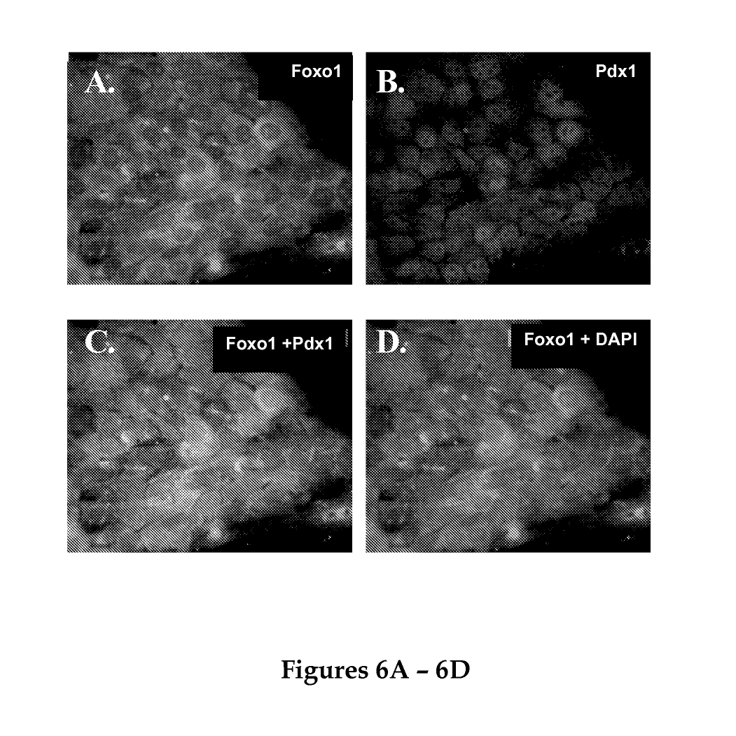

SIPDC were characterized by RT-PCR. SIPDC express duct cell markers (cytokeratin 19 or carbonic anhydrolase II), but do not express endocrine (insulin, glucagon, somatostatin or pancreatic polypeptide) or exocrine (amylase, trypsin or elastase) pancreatic markers. SIPDC express two important transcription factors for endocrine cell differentiation Pdx1 (FIGS. 3 and 6) and Foxo1 (FIG. 6).

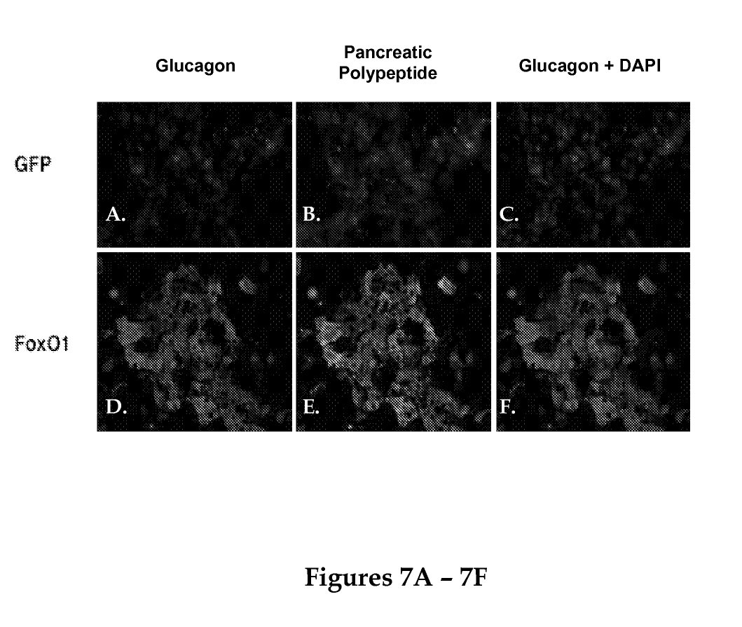

To change the properties of duct cells into hormone-producing endocrine cells, a mutant version of the forkhead transcription factor FoxO1 was introduced (serine 253 of SEQ ID NO:2 was mutated to alanine). After a week in culture, duct cells expressing the mutant Fox01 had begun to express genes that are typical of endocrine cells: Isl1, Nkx6.1 and Nkx2.2, NeurodD1, and several others. The cells were positive for glucagon and pancreatic polypeptide by immunohistochemistry. The cells do not express insulin, nor somatostatin (FIG. 7). These results show that SIPDC may act as progenitors of pancreatic endocrine cells.

### Example 2

### Conversion of Spontaneously Immortalized Pancreatic Duct Cells into Hormone-Producing Endocrine Cells

To change the properties of duct cells into hormone-producing endocrine cells, a mutant version of the forkhead transcription factor Fox01 was introduced into the cells. Cells are incubated in serum-free culture medium for 16 hours. A 1 cc solution containing packaged adenovirus is then added for 1 hour. Thereafter, the solution is removed, cells are washed 3× with saline solution and complete culture medium containing 10% serum is added. The mutant Fox01 protein included alanine at position 253 instead of the wild-type serine at position 253 of SEQ ID NO:2. Transfection was accomplished using an adenovirus. After a week in culture, duct cells expressing the mutant Fox01 had begun to express genes that are typical of endocrine cells: Isl1, Nkx6.1 and Nkx2.2, NeurodD1, and several others. The cells were also positive for glucagon and pancreatic polypeptide by immunohistochemistry. The cells do not express insulin or somatostatin (FIG. 7). These results show that the spontaneously immortalized pancreatic duct cells act as progenitors of pancreatic endocrine cells.

### Example 3

### Regulation of Juxta-Ductal Beta Cell Formation by FoxO1 in Pancreatic Development

Diabetes is characterized by complete or relative deficiency of insulin-producing β cells (Accili, 2004; Taniguchi et al., 2006). The growing societal and public health toll of the disease provides impetus to isolate or generate β cells for cellular replacement purposes. Moreover, given that most of the newly found diabetes susceptibility genes appear to affect β cell function, rather than insulin action (Frayling et al., 2007; Grant et al., 2006; Scott et al., 2007; Sladek et al., 2007; Steinthorsdottir et al., 2007; Zeggini et al., 2007); that the two newest classes of antidiabetic medications are β-tropic (Baggio and Drucker, 2006); and that the main therapeutic failures in diabetes are seen in response to β-tropic agents (Kahn et al., 2006; U.K. Prospective Diabetes Study Group, 1995), studies of β cell biology have wide-ranging implications beyond the replacement issue.

Two approaches to β cell generation have been championed: one endeavors to define culture conditions conducive to embryonic stem cell (ES) differentiation into β cells (D'Amour et al., 2006), while the other is based on the hypothesis that endocrine cell progenitors, often identified with duct epithelial cells, exist in the adult pancreas and can yield functional β cells (Bonner-Weir et al., 2000; Seaberg et al., 2004).

Lineage-tracing studies indicate that pancreatic endocrine cells arise from a Neurog3-expressing progenitor pool set aside early in embryonic development (Gu et al., 2002), and that post-natal β cell turnover is a result of limited β cell replication and apoptosis (Dor et al., 2004; Okamoto et al., 2006; Teta et al., 2005). These data point to a limited role of pancreatic duct cells in the maintenance of β cell mass through neogenesis from non-β cell precursors. Nonetheless, these studies do not exclude the possibility of generating endocrine cells via commandeering developmental pathways at the genetic level. Along these lines, this Example describes the observation of a rare population of juxta-ductal Fox01+ cells that do not express insulin. This finding, coupled with the role of FoxOs in governing developmental processes in diverse lineages and in the long-term stability of various tissues (Kitamura et al., 2007; Nakae et al., 2003; Tothova et al., 2007), prompted studies to examine whether these cells are progenitors of duct-associated β cells. This Example describes the use of a combination of developmental, genetic and cell biology analyses to identify, isolate and functionally characterize these cells.

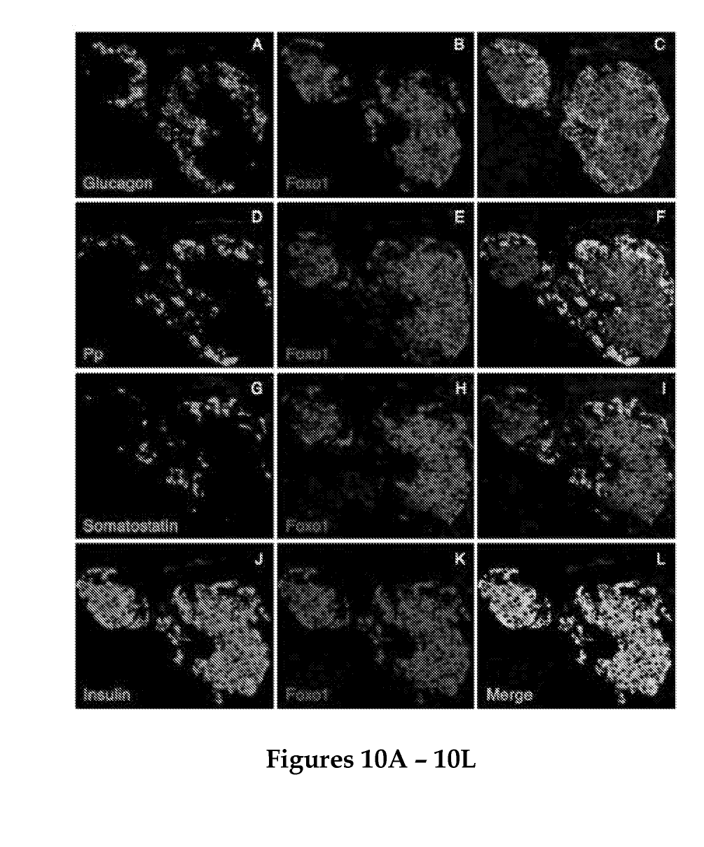

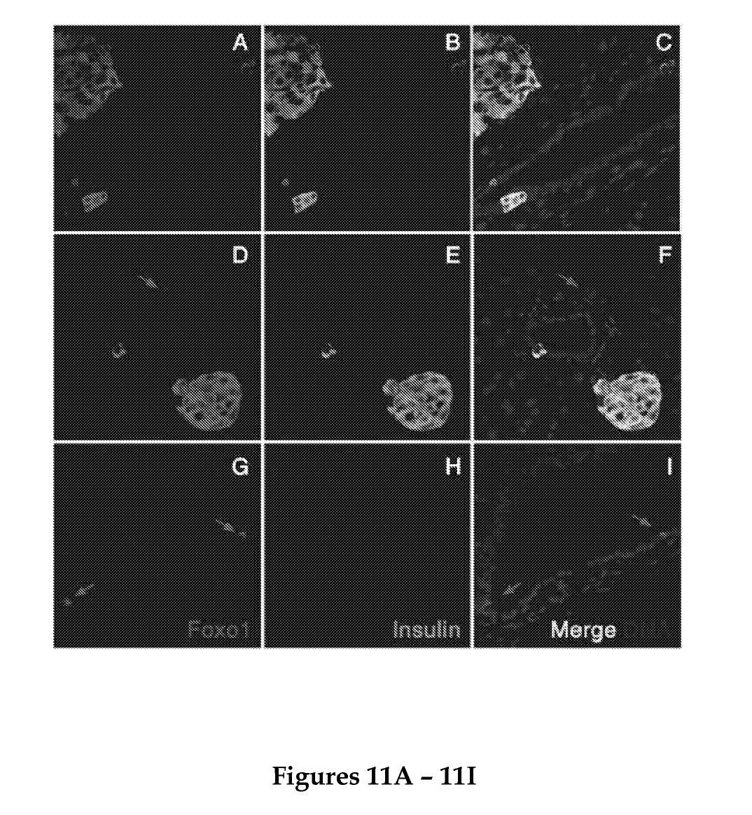

Juxta-ductal FoxO1+ cells in adult mouse pancreas. FoxO1 expression is restricted to pancreatic β cells of the adult pancreas (FIG. 10), including those β cells abutting on ducts (FIGS. 11A-C) (see also Kitamura et al., 2002). In addition, consistent with lineage-tracing data (Kitamura et al., 2002), there are occasional cells near or within ducts that express Fox01, but not insulin (FIGS. 2D-I, arrows). The studies described in this Example were designed to determine whether Fox01 is a marker of a rare sub-population of adult pancreatic cells (henceforth, FoxO1+Ins−) with endocrinogenic potential and to determine whether Fox01 may be involved in the regulation of pancreatic cell fate specification.

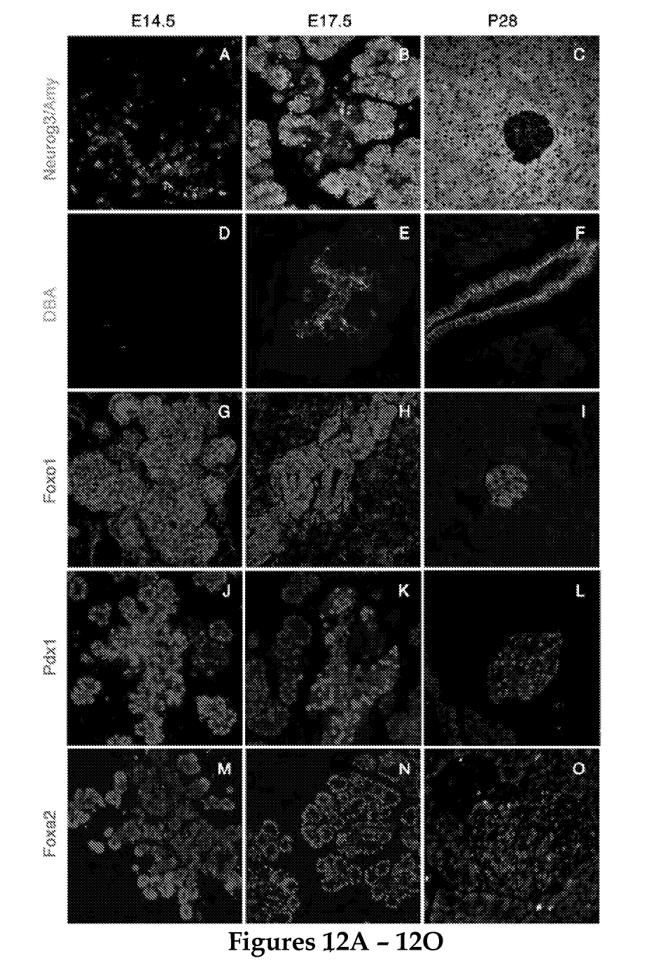

Developmental analysis of FoxO1 expression in the pancreas. As a first step in assessing Fox01 in the pancreas developmental program, Fox01 expression was assessed during pancreatogenesis in the mouse (FIG. 12). To define various lineages, immunohistochemistry was employed using well-characterized reagents, including anti-Amylase antibodies to identify exocrine acinar cells (Amy+) (FIGS. 12A-C), anti-Gfp antibodies (in Neurog3-Gfp transgenic mice) (Murtaugh et al., 2003) to identify endocrine progenitors (Neurog3+) (FIGS. 12A-C), and DBA to identify ductal cells (FIGS. 12E-F). Comparison of expression patterns at E14.5, E17.5 and P28 revealed progressive restriction of the different cell type markers. Fox01 was widely expressed at E14.5 (FIG. 12G), but became restricted at E17.5 (FIG. 12H) and was confined to β cells post-natally (FIG. 12I). This pattern of expression parallels Pdx1 expression, with the notable difference that Fox01 is cytoplasmic and Pdx1 nuclear (FIGS. 12J-L). Fox01 appeared to be nuclear in a subset of cells at E17.5 (FIG. 12H). The related forkhead protein Foxa2 (Lee et al., 2005) was enriched in the tip region of the developing pancreas at E14.5 (FIG. 3M), and remained subsequently expressed in both endocrine and (to a lesser extent) exocrine compartments (FIGS. 12N-O).

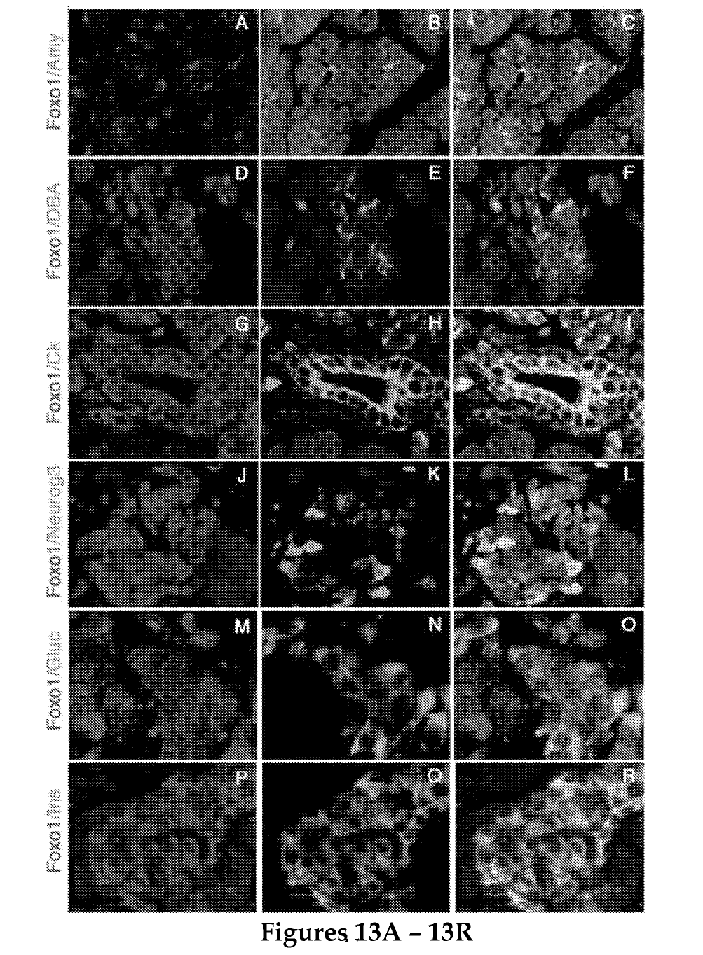

The apparent changes in the distribution and sub-cellular localization of FoxO1 at E17.5 prompted investigation of its co-localization with markers of different pancreatic lineages at this stage. In Amy+ cells (exocrine lineage), Fox01 was exclusively nuclear (FIGS. 13A-C). In DBA+ cells, FoxO1 localized to both nucleus and cytoplasm in a punctate pattern that likely reflects targeting to nuclear Pml bodies (Kitamura et al., 2005), as well as lysosomal compartments (Plas and Thompson, 2003) (FIGS. 13D-F). Identical results were obtained using cytkeratin as a surrogate ductal marker (FIGS. 13G-I). Heterogeneous sub-cellular distribution was also observed in Neurog3+ endocrine progenitors (FIGS. 13J-L). Within the endocrine compartment, Fox01 showed a punctate nuclear pattern in a cells (FIGS. 13M-O), and its signature cytoplasmic pattern in β cells (FIGS. 13P-R). These results show that FoxO1 becomes developmentally restricted to endocrine cells and that, in the process, nuclear localization precedes extinction of Fox01 expression.

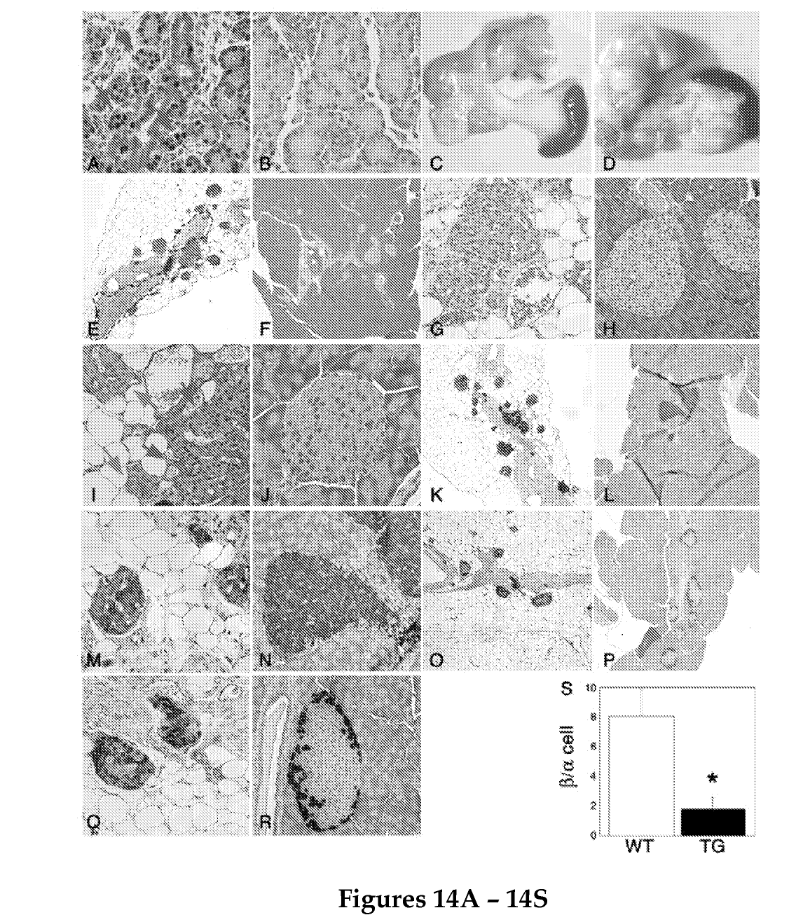

Constitutive nuclear expression of FoxO1 impairs exocrine pancreas development. To determine whether Fox01 nuclear translocation affects pancreatic lineage specification, transgenic mice were generated expressing a FLAG-tagged, constitutively nuclear FoxO1 mutant (FoxO1 ADA) (Nakae et al., 2001) from the Pdx1 promoter (Murtaugh et al., 2003). Timing and extent of transgene expression varied in different animals, as reported with this promoter (Gannon et al., 2000; Heller et al., 2001). In some mice, extensive expression in embryos was observed (FIGS. 14A-B), while in others expression was only detected in adult β cells. The widespread embryonic transgene expression profile was associated with marked pancreatic hypoplasia (FIGS. 14C-D) and extensive disruption of pancreatic architecture. Only remnants of exocrine tissue could be seen in adult mice (FIGS. 14E-F); and endocrine islets showed expanded vascular bed and increased number of a cells (FIGS. 14G-S). Analysis of transgene expression in adult mice showed an admixture of transgenenegative and transgene-positive islets (FIG. 141).

These findings indicate that premature nuclear expression of Fox01 in pancreatic progenitors prevents exocrine cell differentiation and alters β/α cell ratio and islet vasculature, effectively phenocopying the abnormalities of pancreas development seen in mice lacking both insulin and IGF-1 receptors (Kido et al., 2002). The increase in a cell number (FIG. 14S) is consistent with the observation that premature endocrine differentiation drives preferentially the formation of glucagon+ cells (Johansson et al., 2007). The phenotype of Pdx-FoxO1 ADA mice resembles that of Notch gain-of-function (Apelqvist et al., 1999; Murtaugh et al., 2003), and supports the idea that Fox01 cooperates with Notch in cellular differentiation (Kitamura et al., 2007).

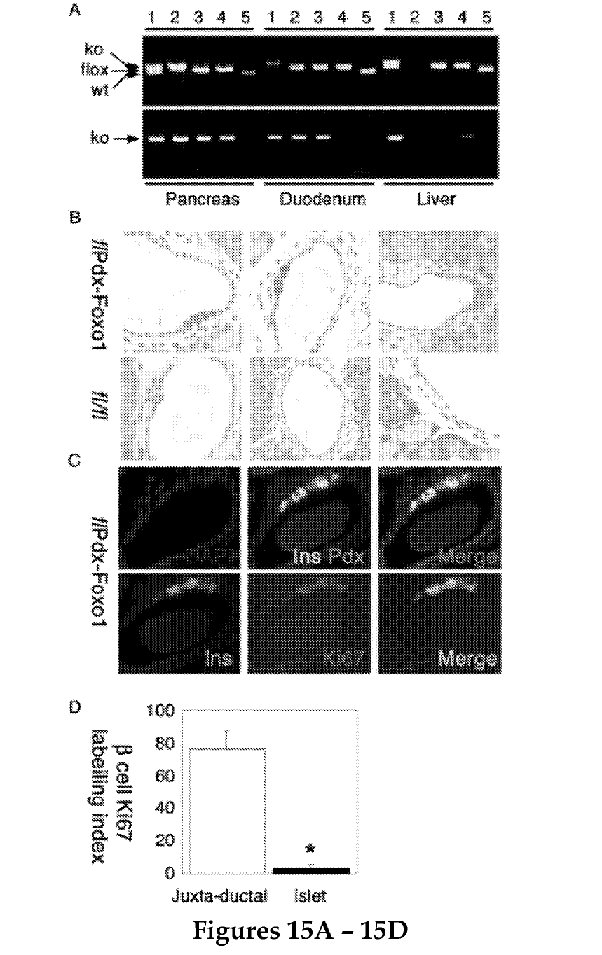

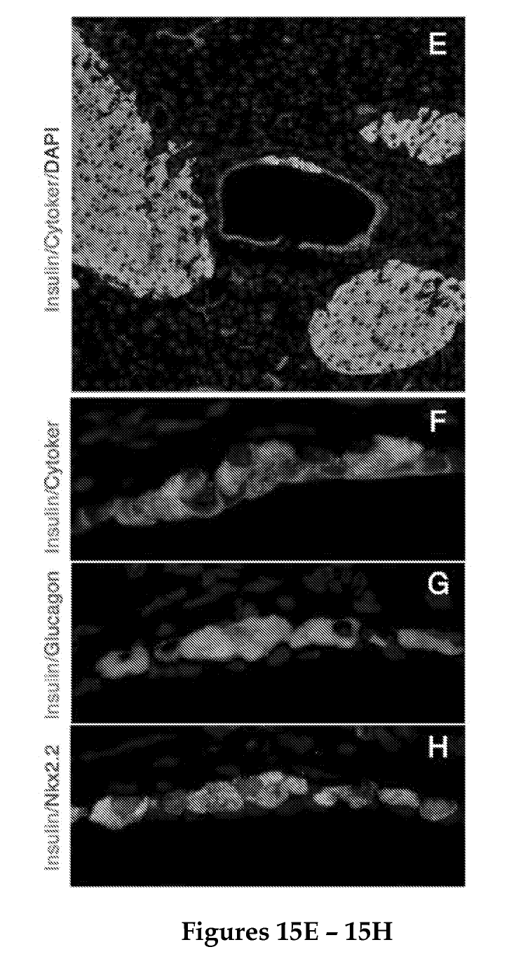

Generation and analysis of FoxO1 conditional knockouts in pancreas. The data in transgenic mice indicate that the timing of Fox01 nuclear translocation is critical for terminal differentiation of pancreatic lineages. When viewed in the context of this study on the role of juxta-ductal FoxO+Ins− cells, the findings indicate that these cells represent remnants of an uncommitted progenitor population in the adult pancreas. Studies were designed to determine the effects of loss of Fox01 function at different stages of pancreatogenesis, using intercrosses of Pdx1(cre), Neurog3(cre) or Ins(cre) transgenics with mice bearing floxed Fox01 alleles (Paik et al., 2007). In Pdx1(cre):FoxO1^{-/-} offspring, FoxO1 should be ablated in all pancreatic cell types (Gu et al., 2002), while in Neurog3(cre):FoxO1^{-/-} mice, ablation should occur in all enteroendocrine cells (Schonhoffet al., 2004); and, in Ins(cre):FoxO1^{-/-} mice, exclusively in β cells (Herrera, 2000). Genotyping of DNA extracted from liver, pancreas and duodenum showed that cre-mediated excision occurred as planned (FIG. 15A). In addition, the lineage targeting of Cre was confirmed by intercrossing cre transgenics with ROSA26-Gfp reporter mice (Kitamura et al., 2006).

If FoxO+Ins-cells are progenitors of juxta-ductal β cells and Fox01 regulates their differentiation in a Notch-like manner (i.e., it must be kept inactive during development to prevent premature differentiation), FoxO1 ablation by Pdx1 (cre) should increase the number of juxta-ductal β cells, while ablation at later stages [driven by Neurog3(cre) or Ins (cre)] should not. If the FoxO1+Ins− population is not a precursor of juxta-ductal β cells and/or FoxO1 is a bystander in the differentiation process, no changes to the number of juxta-ductal β cells will be observed. If juxta-ductal β cells are like any other β cell, and do not derive from FoxO1+Ins− cells, but FoxO1 affects β cell differentiation/proliferation, changes in juxta-ductal β cells should mirror those in islet β cells, and the three conditional knockouts will phenocopy each other.

Pancreas morphology and gross anatomical appearance were normal in mice homozygous for the conditional alleles. However, immunohistochemical analyses of pancreata from Pdx1(cre):FoxO1^{-/-} mice showed clusters of two to four insulin+Pdx1+ cells in ˜15% of surveyed pancreatic ducts (FIGS. 15B-C). These cells were absent in Neurog3(cre):FoxO1^{-/-} and Ins(cre):FoxO1^{-/-} mice. In control FoxO^{flox/flox} mice, insulin+ cells were not detected in the duct proper, even when islets were located near ducts (FIG. 15B). The Ki67 labeling index of juxta-ductal insulin+ cells was 20-fold higher than islet β cells (FIGS. 15C-D), indicating that they replicate at a significantly higher rate than islet β cells. If the juxta-ductal insulin+ cells seen in Pdx(cre):FoxO1^{-/-} mice were simply β cells near ducts, they should be observed in all three conditional knockouts, and their replication rates should be the same as islet β cells. Further immunohistochemistry with anti-insulin and anti-cytokeratin antisera indicated that insulin+ cells were juxtaposed to, but distinct from, duct epithelial cells (FIGS. 6E-F). The cells were Vimentin− and Glucagon− (FIG. 15G), but Nkx2.2+(FIG. 15H), consistent with a β cell identity.

The presence of relatively large numbers of bona fide juxta-ductal β cells following FoxO1 ablation in pancreatic progenitors indicates that these cells arise from the FoxO+Ins− sub-population. Alternatively, FoxO1 ablation in pancreatic progenitors alters the ductal microenvironment, either generating a ductal homing signal for β cells, or promoting 13 cell differentiation of juxta-ductal progenitors, distinct from FoxO+Ins− cells.

Generation of primary cultures of FoxO1+Ins− cells. The identification of this unique FoxO1+Ins− cellular sub-population, together with the sharp increase of insulin+juxtaductal cells seen in Pdx1(cre):FoxO1^{-/-} mice, led to the design of experiments to test whether FoxO1+Ins− cells possess endocrine progenitor features when cultured.

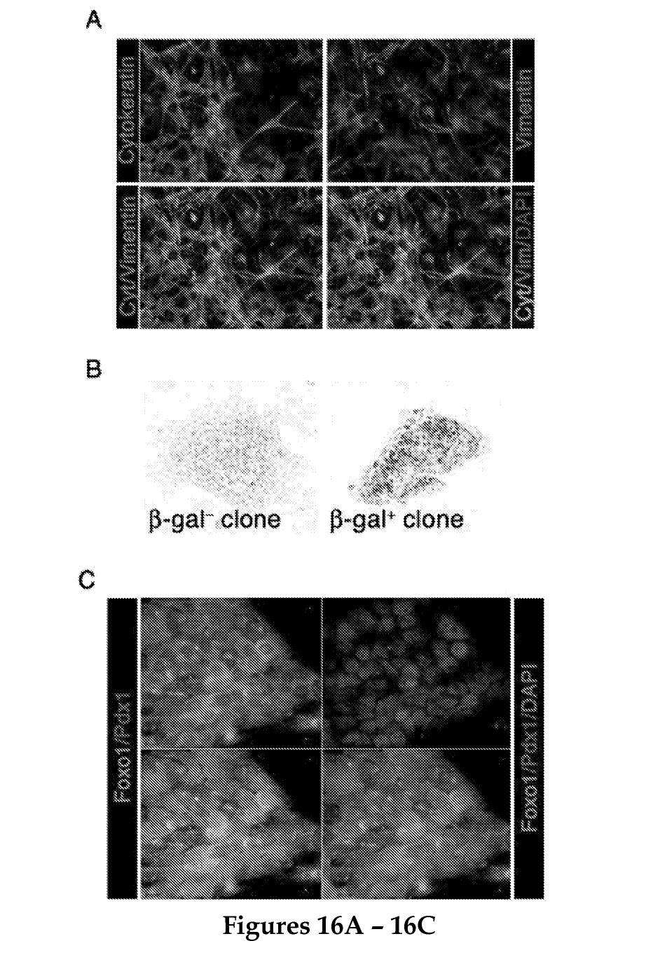

To identify and isolate FoxO1+ cells, a genetic selection approach was used, relying on a reporter β-gal gene targeted to the FoxO1 locus (Hosaka et al., 2004). Primary pancreatic cell preparations were cultured in defined serum-free conditions (Seaberg et al., 2004; Yamamoto et al., 2006), after removing endocrine islets by filtration. (Also see Example 1.) After seven-day culture, immunocytochemistry with epithelial (cytokeratin) and mesenchymal markers (vimentin) revealed different sub-populations of cells: cobblestone-shaped, Ck+ cells (FIG. 16A, green); spindle-shaped, Vm+ cells (FIG. 16A, red); Ck+Vm+double-positive cells (FIG. 16A, yellow); and Ck−Vm− cells (FIG. 16, blue nuclei with unstained cytoplasm). These data indicate that the culture is heterogeneous in nature and includes mixedlineage cells (Ck+Vm+), potentially undergoing epithelial-mesenchymal transition (Gershengorn et al., 2004).

Single cell cloning was performed by limiting dilution, and individual clones were stained with X-gal to identify those derived from FoxO1+ cells. Clonal β-gal+ and β3-gal− cells were obtained (FIG. 16B). Twenty-four β-gal+clones were isolated. As expected, all expressed FoxO1 in the cytoplasm and nucleolus (FIG. 16C, green), as well as nuclear Pdx1 (FIG. 16C, red) (Kitamura et al., 2002).

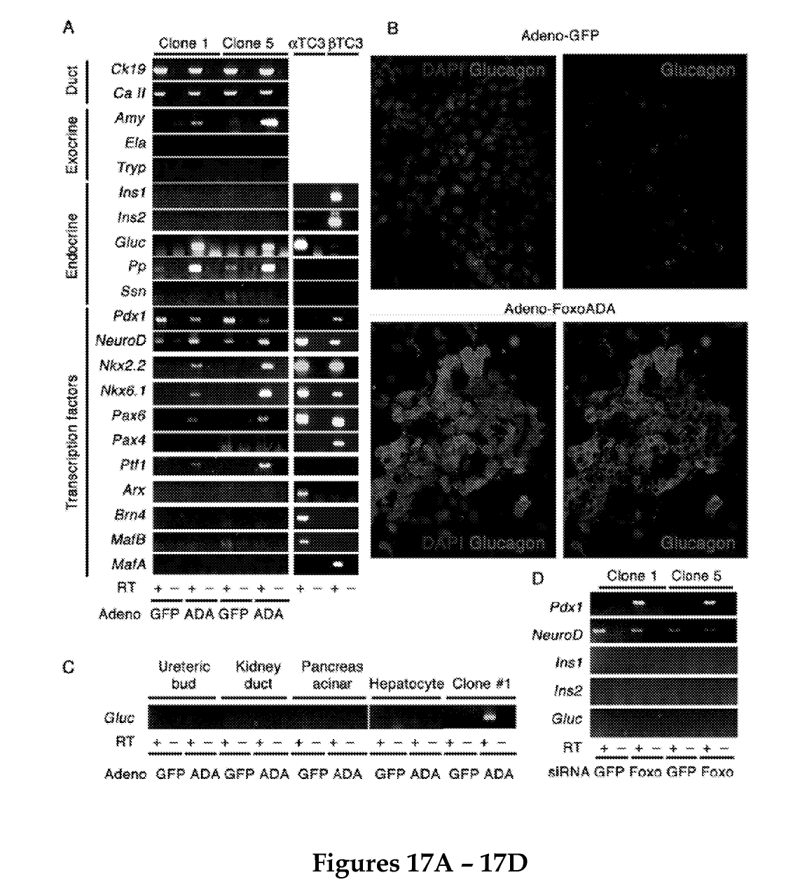

Based on the finding that FoxO1 nuclear expression in transgenic mice favors the adoption of an endocrine fate, experiments were designed to test whether the clonal isolates of FoxO+ cells could be differentiated into endocrine cells by FoxO1 gain-of-function, using adenoviral transduction of FoxO1ADA (constitutively nuclear). Representative results in two clones are shown in FIG. 17. In basal conditions, cells express ductal markers Cytokeratin19 (Ck19) and Carbonic anhydrase II (CaII), but none of the exocrine (Amylase, Elastase, Trypsin) and endocrine markers (Insulin1, Insulin2, Glucagon, Pancreatic polypeptide, Somatostatin). Among pancreas-specific or -enriched transcription factors, they express Pdx1 and NeuroD (FIG. 17A). Expression of FoxO1 ADA had no effect on Ck19 and CaII, but induced Amy, Gluc, and PP (FIG. 17A). Importantly, FoxO1 ADA did not induce Ins1, Ins2, Ssn, Ela and Try (FIG. 17A). These data are consistent with the increased number of a cells in Pdx-FoxO1ADA transgenic mice (FIG. 14). Glucagon expression was confirmed by immunocytochemistry with anti-glucagon antibody (FIG. 17B). The effect of FoxO1 ADA on transcription factors required for pancreatic development and cell type specification was assessed. Transduction of FoxO1 ADA decreased Pdx1 and increased NeuroD (Kitamura et al., 2002; Kitamura et al., 2005). FoxO1 ADA induced expression of Nkx2.2, Nkx6.1, Pax6 and Ptf1, but not Pax4, Arx, Brn4, MafB or MafA (FIG. 17A). The resulting expression pattern is not typical of a cells. It indicates that, although FoxO1 ADA is able to induce Glucagon and Pp expression, these cells are unlike bona fide a cells.

To test whether FoxO1-induced glucagon expression is specific to FoxO+Ins− pancreatic cells or is commonly seen in other duct-derived murine cell lines, UB (ureteric bud-derived kidney duct) (Barasch et al., 1996), M-1 (kidney cortical collecting duct) (Stoos et al., 1991), or TGP47 cells (pancreatic adenocarcinoma with ductal features) (Pettengill et al., 1994) were transduced with FoxO1 ADA, and SV40-transformed hepatocytes were used as negative control (Rother et al., 1998). The results show that FoxO1 ADA induced Glucagon only in FoxO1+Ins− derived clones, indicating that this effect of FoxO1 is specific for these cells (FIG. 17C). Prolonged culture resulted in a time-dependent loss of glucagon immunoreactivity in 7-10 days.

Since FoxO1 ablation in Pdx(cre):FoxO1^{-/-} mice resulted in increased Insulin+juxta-ductal cells (FIG. 7B), studies were designed to test whether FoxO1 knockdown would promote Ins1 or Ins2 expression in FoxO+Ins− cells. FoxO1 siRNA resulted in the predicted increase of Pdx1 (Kitamura et al., 2002) and decrease of NeuroD (Kitamura et al., 2005), but induction of Ins1, Ins2, and Gluc transcription was not detected (FIG. 17D). Additional culture conditions were tested that have been employed to differentiate clonal adult pancreatic cells into β-like cells (Seaberg et al., 2004), but were unable to detect Ins1 or Ins2 expression.

FoxO1's role in pancreatic development. The genetic, developmental and cell biology analyses described in this Example show a permissive role of FoxO1 in exocrine pancreas differentiation, loosely reminiscent of its role in adipocytes (Nakae et al., 2003); and a pro-endocrine role in pancreatic progenitors, prior to the divergence of endocrine, exocrine and ductal lineages. Constitutive activation, in transgenic mice and in primary cultures of FoxO+Ins− cells, drives preferentially the a cell phenotype, similar to Neurogenin-3 activation (Johansson et al., 2007). FoxO1 ablation in pancreatic, but not in endocrine progenitors or differentiated β cells, specifically increases juxta-ductal β cells. Thus, the timing of FoxO1 activation appears critical for terminal differentiation of specific endocrine cell types. A potential mechanism by which FoxO1 ablation promotes endocrine differentiation is through its interaction with Notch signaling (Kitamura et al., 2007). Like FoxO1 ablation, Notch ablation results in higher number of endocrine cells only when the gene is inactivated in pancreatic progenitors, but not in differentiated endocrine cells (Murtaugh et al., 2003). The fact that FoxO1 deletion promotes β cell formation in cells adjacent to pancreatic duct epithelia suggests that, in this context, β cell differentiation is dependent on local cues, for example growth or differentiation factors released from duct-associated cells.

What is the physiologic role of FoxO+Ins− adult pancreatic cells? There is disagreement as to whether ductal cells undergo endocrine differentiation in the adult pancreas (Bonner-Weir et al., 2000; Dor et al., 2004; Gu et al., 2002; Yatoh et al., 2007). The clones of FoxO+Ins− cells characterized in this Example seemingly engage in a limited endocrine-like differentiation program in vitro. Some studies show a limited role for neogenesis in β cell turnover (Okamoto et al., 2006; Dor et al., 2004; Lin et al., 2004; Nir et al., 2007; Teta et al., 2005). Nonetheless, this Example provides proof-of-principle that FoxO+Ins− cells have unique properties that could be exploited for cellular replacement purposes. It is not yet known whether FoxO+Ins− cells are the same cells identified in clonal studies of adult pancreatic endocrine precursors (Seaberg et al., 2004). Studies in cultured cells dovetail with two in vivo findings: the a cell-like phenotype brought about by FoxO1 gain-of-function is consistent with the notion that premature activation of endocrine differentiation preferentially yields glucagon+ cells (Johansson et al., 2007). Similarly, co-activation of Glucagon and Pp expression by FoxO1 is reminiscent of the phenotype due to Arx gain-of-function (Collombat et al., 2007). Failure to yield β cells may reflect a critical requirement for mesenchymal/epithelial interactions, as observed in normal pancreatic development (Ahlgren et al., 1996; Gittes et al., 1996; Miralles et al., 1999; Miralles et al., 1998). Further studies in this area may include co-culture experiments, as well as isolation of FoxO1+Ins− cells from embryonic pancreata.

The results presented in this Example demonstrate that FoxO1 ablation increases β cell formation at a specific anatomical location and during a narrow developmental window; showing that β cells can be generated from sources other than ES cells.

The following exemplary materials and methods may be used in connection with the methods disclosed herein:

Antibodies and immunohistochemistry. The following antibodies were used: anti-Pancytokeratin (Sigma), anti-Vimentin (Santa-Cruz), anti-Nkx2.2 (Hybridoma Bank, University of Iowa), anti-FoxO1 (Kitamura et al., 2006), anti-Pdx1 (Kitamura et al., 2002), anti-glucagon (Sigma), anti-insulin (DAKO), anti-somatostatin (Chemicon), antipancreatic polypeptide (Linco), anti-amylase (ABCAM), anti-GFP (Santa-Cruz), and anti-FoxA2 (Sasaki and Hogan, 1994). Fluorescent-conjugated DBA (0.05 mg/ml, Vector Laboratories) was used for duct cell staining (Kobayashi et al., 2002). Immunostaining was performed using 5 μm-thick paraffin sections and, in some experiments, antigen retrieval, as described in Kitamura et al., 2002. Immune complexes were visualized with FITC- or CY3-conjugated secondary antibodies. To quantitate insulin+juxta-ductal cells, ducts were scored with insulin+ cells on each section as percentage of the total number of anatomically identifiable ducts. Six sections were scored for each mouse, and six mice for each genotype. The Ki67-labeling index of islet β cells and juxta-ductal insulin+ cells was determined by dividing the number of Ki67+ cells by the total number of islet β cells or juxta-ductal insulin+ cells in at least four sections in six Pdx1(cre):FoxO11 mice.

Cells. βTC3, αTC3 (Efrat et al., 1988), UB (embryonic ureteric bud) (Barasch et al., 1996), M-1 (SV40-transformed kidney cortical collecting duct) (Stoos et al., 1991), TGP-47 (pancreatic acinar carcinoma) (Pettengill et al., 1994) and SV40-transformed hepatocytes have been described (Rother et al., 1998).

Animal generation and analysis. Pdx1(cre) (Gu et al., 2002), Neurog3(cre) (Schonhoff et al., 2004), Ins (cre) (Herrera, 2000), and FoxO1flox mice have been described (Paik et al., 2007). Pdx-FoxO1ADA transgenic mice were generated by microinjection into fertilized zygotes of a construct encoding FLAG-tagged FoxO1 ADA cDNA (Nakae et al., 2001) driven the 4.5 kb Pdx1 promoter with β-globin intron and polyA signal (Stoffers et al., 1999). Two founder lines were characterized and used for the studies described. PCR genotyping was carried out with primers: GCTTAGAGCAGAGATGTTCTCACATT (SEQ ID NO: 3); CCAGAGTCTTTGTATCAGGCAAATAA (SEQ ID NO:4); CAAGTCCATTAATTCAGCACATTGA (SEQ ID NO:5). Standard mRNA isolation and real-time RT-PCR techniques were used.

Primary culture and cloning of pancreatic cells. Pancreata were dissected from 2-month old mice, the isolated tissue was digested in 1 ml of M199 medium containing 1 mg/ml collagenase P (Roche) and diluted the cellular aggregates in 30 ml of the same medium (Kitamura et al., 2001). After filtration through a Spectra-mesh (408 μm; Spectrum Laboratories), the cell aggregates were resuspended in RPMI supplemented with 10% FCS, 5.5 mM glucose, 100 mg/ml penicillin, 100 mg/ml streptomycin and 250 ng/ml amphotericin B, and cultured them at 37° C. in 5% CO2. After seven days, the medium was replaced with serum-free RPMI supplemented with 8 mM glucose, 1 g/l ITS (5 mg/l insulin, 5 mg/l transferring, and 5 mg/l selectin), 2 g/l BSA, 10 mM nicotinamide and 10 ng/ml keratinocyte growth factor (all from Sigma). The serum-free medium was replaced every third day during the selection process. The resulting cells showed stable growth in serum-free medium, and were cloned by limiting dilution in 96-well plates.

Adenovirus and siRNA transfection. See Kitamura et al., 2007 for GFP, FoxO1 ADA adenovirus and siRNA methods.

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention.

### REFERENCES

Accili, D. (2004). Lilly lecture 2003: the struggle for mastery in insulin action: from triumvirate to republic. Diabetes 53, 1633-1642.
Ahlgren, U., Jonsson, J., and Edlund, H. (1996). The morphogenesis of the pancreatic mesenchyme is uncoupled from that of the pancreatic epithelium in IPF1/PDX1-deficient mice. Development 122, 1409-1416.
Apelqvist, A., Li, H., Sommer, L., Beatus, P., Anderson, D. J., Honjo, T., Hrabe de Angelis, M., Lendahl, U., and Edlund, H. (1999). Notch signalling controls pancreatic cell differentiation. Nature 400, 877-881.
Baggio, L. L., and Drucker, D. J. (2006). Therapeutic approaches to preserve islet mass in type 2 diabetes. Annu Rev Med 57, 265-281.
Barasch, J., Pressler, L., Connor, J., and Malik, A. (1996). A ureteric bud cell line induces nephrogenesis in two steps by two distinct signals. Am J Physiol 271, F50-61.
Bonner-Weir, S., Taneja, M., Weir, G. C., Tatarkiewicz, K., Song, K. H., Sharma, A., and O'Neil, J. J. (2000). In vitro cultivation of human islets from expanded ductal tissue. Proc Natl Acad Sci USA 97, 7999-8004.
Collombat, P., Hecksher-Sorensen, J., Krull, J., Berger, J., Riedel, D., Herrera, P. L., Serup, P., and Mansouri, A. (2007). Embryonic endocrine pancreas and mature beta cells acquire alpha and PP cell phenotypes upon Arx misexpression. J Clin Invest 117, 961-970.
D'Amour, K. A., Bang, A. G., Eliazer, S., Kelly, O. G., Agulnick, A. D., Smart, N. G., Moorman, M. A., Kroon, E., Carpenter, M. K., and Baetge, E. E. (2006). Production of pancreatic hormone-expressing endocrine cells from human embryonic stem cells. Nat Biotechnol 24, 1392-1401.
Dietrich, P., Dragatsis, I., Xuan, S., Zeitlin, S., and Efstratiadis, A. (2000). Conditional mutagenesis in mice with heat shock promoter-driven cre transgenes. Mammalian Genome 11, 196-205.
Dor, Y., Brown, J., Martinez, O. I., and Melton, D. A. (2004). Adult pancreatic betacells are formed by self-duplication rather than stem-cell differentiation. Nature 429, 41-46.
Efrat, S., Linde, S., Kofod, H., Spector, D., Delannoy, M., Grant, S., Hanahan, D., and Baekkeskov, S. (1988). Beta-cell lines derived from transgenic mice expressing a hybrid insulin gene-oncogene. Proc Natl Acad Sci USA 85, 9037-9041.
Frayling, T. M., Timpson, N. J., Weedon, M. N., Zeggini, E., Freathy, R. M., Lindgren, C. M., Perry, J. R., Elliott, K. S., Lango, H., Rayner, N. W., et al. (2007). A common variant in the FTO gene is associated with body mass index and predisposes to childhood and adult obesity. Science 316, 889-894.
Gannon, M., Herrera, P. L., and Wright, C. V. (2000). Mosaic Cre-mediated recombination in pancreas using the pdx-1 enhancer/promoter. Genesis 26, 143-144.
Gershengorn, M. C., Hardikar, A. A., Wei, C., Geras-Raaka, E., Marcus-Samuels, B., and Raaka, B. M. (2004). Epithelial-to-mesenchymal transition generates proliferative human islet precursor cells. Science 306, 2261-2264.
Gittes, G. K., Galante, P. E., Hanahan, D., Rutter, W. J., and Debase, H. T. (1996). Lineage-specific morphogenesis in the developing pancreas: role of mesenchymal factors. Development 122, 439-447.
Grant, S. F., Thorleifsson, G., Reynisdottir, I., Benediktsson, R., Manolescu, A., Sainz, J., Helgason, A., Stefansson, H., Emilsson, V., Helgadottir, A., et al. (2006). Variant of transcription factor 7-like 2 (TCF₇L2) gene confers risk of type 2 diabetes. Nat Genet. 38, 320-323.
Gu, G., Dubauskaite, J., and Melton, D. A. (2002). Direct evidence for the pancreatic lineage: NGN3+ cells are islet progenitors and are distinct from duct progenitors. Development 129, 2447-2457.
Heller, R. S., Stoffers, D. A., Bock, T., Svenstrup, K., Jensen, J., Horn, T., Miller, C. P., Habener, J. F., Madsen, O. D., and Serup, P. (2001). Improved glucose tolerance and acinar dysmorphogenesis by targeted expression of transcription factor PDX-1 to the exocrine pancreas. Diabetes 50, 1553-1561.
Herrera, P. L. (2000). Adult insulin- and glucagon-producing cells differentiate from two independent cell lineages. Development 127, 2317-2322.
Hosaka, T., Biggs, W. H., 3rd, Tieu, D., Boyer, A. D., Varki, N. M., Cavenee, W. K., and Arden, K. C. (2004). Disruption of forkhead transcription factor (FOXO) family members in mice reveals their functional diversification. Proc Natl Acad Sci USA 101, 2975-2980.
Johansson, K. A., Dursun, U., Jordan, N., Gu, G., Beermann, F., Gradwohl, G., and Grapin-Botton, A. (2007). Temporal control of neurogenin3 activity in pancreas progenitors reveals competence windows for the generation of different endocrine cell types. Dev Cell 12, 457-465.
Kahn, S. E., Haffner, S. M., Heise, M. A., Herman, W. H., Holman, R. R., Jones, N. P., Kravitz, B. G., Lachin, J. M., O'Neill, M. C., Zinman, B., et al. (2006). Glycemic durability of rosiglitazone, metformin, or glyburide monotherapy. N Engl J Med 355, 2427-2443.
Kido, Y., Nakae, J., Hribal, M. L., Xuan, S., Efstratiadis, A., and Accili, D. (2002). Effects of Mutations in the Insulin-like Growth Factor Signaling System on Embryonic Pancreas Development and beta-Cell Compensation to Insulin Resistance. J Biol Chem 277, 36740-36747.
Kitamura, T., Feng, Y., Kitamura, Y. I., Chua, S. C., Jr., Xu, A. W., Barsh, G. S., Rossetti, L., and Accili, D. (2006). Forkhead protein FoxO1 mediates Agrp-dependent effects of leptin on food intake. Nat Med 12, 534-540.
Kitamura, T., Kido, Y., Nef, S., Merenmies, J., Parada, L. F., and Accili, D. (2001). Preserved pancreatic beta-cell development and function in mice lacking the insulin receptor-related receptor. Mol Cell Biol 21, 5624-5630.
Kitamura, T., Kitamura, Y. I., Funahashi, Y., Shawber, C. J., Castrillon, D. H., Kollipara, R., Depinho, R. A., Kitajewski, J., and Accili, D. (2007). A Foxo/Notch pathway controls myogenic differentiation and fiber type specification. J Clin Invest 117, 2477-2485.
Kitamura, T., Nakae, J., Kitamura, Y., Kido, Y., Biggs, W. H., 3rd, Wright, C. V., White, M. F., Arden, K. C., and Accili, D. (2002). The forkhead transcription factor Foxo1 links insulin signaling to Pdx1 regulation of pancreatic beta cell growth. J Clin Invest 110, 1839-1847.
Kitamura, Y. I., Kitamura, T., Kruse, J. P., Raum, J. C., Stein, R., Gu, W., and Accili, D. (2005). FoxO1 protects against pancreatic beta cell failure through NeuroD and MafA induction. Cell Metab 2, 153-163.
Kobayashi, H., Spilde, T. L., L1, Z., Marosky, J. K., Bhatia, A. M., Hembree, M. J., Prasadan, K., Preuett, B. L., and Gittes, G. K. (2002). Lectin as a marker for staining and purification of embryonic pancreatic epithelium. Biochem Biophys Res Commun 293, 691-697.
Lee, C. S., Sund, N. J., Behr, R., Herrera, P. L., and Kaestner, K. H. (2005). Foxa2 is required for the differentiation of pancreatic alpha-cells. Dev Biol 278, 484-495.
Lin, X., Taguchi, A., Park, S., Kushner, J. A., Li, F., Li, Y., and White, M. F. (2004). Dysregulation of insulin receptor substrate 2 in beta cells and brain causes obesity and diabetes. J Clin Invest 114, 908-916.
Miralles, F., Czemichow, P., Ozaki, K., Itoh, N., and Scharfmann, R. (1999). Signaling through fibroblast growth factor receptor 2b plays a key role in the development of the exocrine pancreas. Proc Natl Acad Sci USA 96, 6267-6272.
Miralles, F., Czernichow, P., and Scharfmann, R. (1998). Follistatin regulates the relative proportions of endocrine versus exocrine tissue during pancreatic development. Development 125, 1017-1024.
Murtaugh, L. C., Stanger, B. Z., Kwan, K. M., and Melton, D. A. (2003). Notch signaling controls multiple steps of pancreatic differentiation. Proc Natl Acad Sci USA 100, 14920-14925.
Nakae, J., Kitamura, T., Kitamura, Y., Biggs, W. H., Arden, K. C., and Accili, D. (2003). The forkhead transcription factor foxo1 regulates adipocyte differentiation. Dev Cell 4, 119-129.
Nakae, J., Kitamura, T., Silver, D. L., and Accili, D. (2001). The forkhead transcription factor Foxo1 (Fkhr) confers insulin sensitivity onto glucose-6-phosphatase expression. J Clin Invest 108, 1359-1367.
Nir, T., Melton, D. A., and Dor, Y. (2007). Recovery from diabetes in mice by beta cell regeneration. J Clin Invest 117, 2553-2561.
Okamoto, H., Hribal, M. L., Lin, H. V., Bennett, W. R., Ward, A., and Accili, D. (2006). Role of the forkhead protein FoxO1 in beta cell compensation to insulin resistance. J Clin Invest 116, 775-782.
Paik, J. H., Kollipara, R., Chu, G., Ji, H., Xiao, Y., Ding, Z., Miao, L., Tothova, Z., Horner, J. W., Carrasco, D. R., et al. (2007). FoxOs Are Lineage-Restricted Redundant Tumor Suppressors and Regulate Endothelial Cell Homeostasis. Cell 128, 309-323.
Pettengill, O. S., Memoli, V. A., Brinck-Johnsen, T., and Longnecker, D. S. (1994). Cell lines derived from pancreatic tumors of Tg(Ela-1-SV40E)Bri18 transgenic mice express somatostatin and T antigen. Carcinogenesis 15, 61-65.
Plas, D. R., and Thompson, C. B. (2003). Akt activation promotes degradation of tuberin and FOXO3a via the proteasome. J Biol Chem 278, 12361-12366.
Rother, K. I., Imai, Y., Caruso, M., Beguinot, F., Formisano, P., and Accili, D. (1998). Evidence that IRS-2 phosphorylation is required for insulin action in hepatocytes. J Biol Chem 273, 17491-17497.
Sasaki, H., and Hogan, B. L. (1994). HNF-3 beta as a regulator of floor plate development. Cell 76, 103-115.
Schonhoff, S. E., Giel-Moloney, M., and Leiter, A. B. (2004). Neurogenin 3-expressing progenitor cells in the gastrointestinal tract differentiate into both endocrine and nonendocrine cell types. Dev Biol 270, 443-454.
Scott, L. J., Mohlke, K. L., Bonnycastle, L. L., Willer, C. J., Li, Y., Duren, W. L., Erdos, M. R., Stringham, H. M., Chines, P. S., Jackson, A. U., et al. (2007). A genome-wide association study of type 2 diabetes in Finns detects multiple susceptibility variants. Science 316, 1341-1345.
Seaberg, R. M., Smukler, S. R., Kieffer, T. J., Enikolopov, G., Asghar, Z., Wheeler, M. B., Korbutt, G., and van der Kooy, D. (2004). Clonal identification of multipotent precursors from adult mouse pancreas that generate neural and pancreatic lineages. Nat Biotechnol 22, 1115-1124.
Sladek, R., Rocheleau, G., Rung, J., Dina, C., Shen, L., Serre, D., Boutin, P., Vincent, D., Belisle, A., Hadjadj, S., et al. (2007). A genome-wide association study identifies novel risk loci for type 2 diabetes. Nature 445, 881-885.
Steinthorsdottir, V., Thorleifsson, G., Reynisdottir, I., Benediktsson, R., Jonsdottir, T., Walters, G. B., Styrkarsdottir, U., Gretarsdottir, S., Emilsson, V., Ghosh, S., et al. (2007). A variant in CDKAL1 influences insulin response and risk of type 2 diabetes. Nat Genet. 39, 770-775.
Stoffers, D. A., Heller, R. S., Miller, C. P., and Habener, J. F. (1999). Developmental expression of the homeodomain protein IDX-1 in mice transgenic for an IDX-1 promoter/lacZ transcriptional reporter. Endocrinology 140, 5374-5381.
Stoos, B. A., Naray-Fejes-Toth, A., Carretero, O. A., Ito, S., and Fejes-Toth, G. (1991). Characterization of a mouse cortical collecting duct cell line. Kidney international 39, 1168-1175.
Taniguchi, C. M., Emanuelli, B., and Kahn, C. R. (2006). Critical nodes in signaling pathways: insights into insulin action. Nature reviews 7, 85-96.
Teta, M., Long, S. Y., Wartschow, L. M., Rankin, M. M., and Kushner, J. A. (2005). Very slow turnover of beta-cells in aged adult mice. Diabetes 54, 2557-2567.
Tothova, Z., Kollipara, R., Huntly, B. J., Lee, B. H., Castrillon, D. H., Cullen, D. E., McDowell, E. P., Lazo-Kallanian, S., Williams, I. R., Sears, C., et al. (2007). FoxOs Are Critical Mediators of Hematopoietic Stem Cell Resistance to Physiologic Oxidative Stress. Cell 128, 325-339.
U.K. Prospective Diabetes Study Group (1995). U.K. prospective diabetes study 16. Overview of 6 years' therapy of type II diabetes: a progressive disease. Diabetes 44, 1249-1258.
Yamamoto, T., Yamato, E., Taniguchi, H., Shimoda, M., Tashiro, F., Hosoi, M., Sato, T., Fujii, S., and Miyazaki, J. I. (2006). Stimulation of cAMP signalling allows isolation of clonal pancreatic precursor cells from adult mouse pancreas. Diabetologia 49, 2359-2367.
Yatoh, S., Dodge, R., Akashi, T., Omer, A., Sharma, A., Weir, G. C., and Bonner-Weir, S. (2007). Differentiation of affinity-purified human pancreatic duct cells to beta-cells. Diabetes 56, 1802-1809.
Zeggini, E., Weedon, M. N., Lindgren, C. M., Frayling, T. M., Elliott, K. S., Lango, H., Timpson, N. J., Perry, J. R., Rayner, N. W., Freathy, R. M., et al. (2007). Replication of genome-wide association signals in UK samples reveals risk loci for type 2 diabetes. Science 316, 1336-1341.

What is claimed is:

1. An immortalized pancreatic duct cell derived from a primary adult pancreatic duct epithelial cell culture, wherein the immortalized cell expresses Pdx1 and FoxO1.

2. The cell of claim 1, wherein the cell expresses pancreatic duct cell markers.

3. The cell of claim 2, wherein the pancreatic duct cell markers comprise cytokeratin 16 and carbonic anhydrase II.

4. The cell of claim 1, wherein the cell does not express endocrine pancreatic markers.

5. The cell of claim 4, wherein the endocrine pancreatic markers comprise insulin, glucagon, somatostatin and pancreatic polypeptide.

6. The cell of claim 1, wherein the cell does not express exocrine pancreatic markers.

7. The cell of claim 6, wherein the exocrine pancreatic markers comprise amylase, trypsin and elastase.

8. The cell of claim 1, wherein the cell is a human cell.

9. An immortalized pancreatic duct cell derived from a primary adult pancreatic duct epithelial cell, wherein the cell expresses mutated FoxO1.

10. The cell of claim 9, wherein the cells express endocrine pancreatic markers.

11. The cell of claim 10, wherein the endocrine pancreatic markers comprise Isl1, Nkx6.1, Nkx2.2, NeurodD1, glucagon and pancreatic polypeptide.

12. The cell of claim 9, wherein the mutated version of FoxO1 contains a loss-of-function mutation.

13. A pancreatic duct cell line designated 24-1 Duct having ATCC Accession No. PTA-6968.

14. A method for treating beta cell failure, the method comprising administering to a subject spontaneously immortalized pancreatic ductal cell line expressing mutated FoxO1.

15. The method of claim 14, wherein the administering comprises infusion, injection, incapsulation, or any combination thereof.

16. The method of claim 14, wherein the administering comprises transplanting a sponge matrix comprising immortalized pancreatic ductal cells expressing mutated FoxO1 or Spontaneously Immortalized Pancreatic Duct Cells (SIPDC)-derived hormone-producing cells.

17. A method for obtaining a pancreatic duct cell line, the method comprising:
(a) culturing pancreatic duct cells collected from a subject in medium comprising about 10% serum and 5.5 mM glucose for about a week;
(b) culturing the cells in a medium comprising (i) about 8 mM glucose; (ii) about 1 g/L ITS (about 5 mg/l insulin and about 5 mg/l transferrin and about 5 mg/l selectin), (iii) about 2 g/l albumin, (iv) about 10 mM nicotinamide, and (v) about 10 mg/ml keratinocyte growth factor, for about at least another week until the culture comprises nearly all duct cells;
(c) culturing the duct cells with the medium of step (b) further comprising about 10% serum and about 5.5 mM glucose;
(d) passaging the cells of step (c) until the cells' doubling time reach about 24 hours; and
(e) cloning a single cell from the cells of step (d) so as to obtain a pancreatic duct cell line.
